(19) United States
(12) Patent Application Publication
(10) Pub. No.: US 2022/0223245 A1
(43) Pub. Date: Jul. 14, 2022

(54) DRUG RECOMMENDATION METHOD, APPARATUS AND SYSTEM, ELECTRONIC DEVICE AND STORAGE MEDIUM

(71) Applicant: BOE TECHNOLOGY GROUP CO., LTD., Beijing (CN)

(72) Inventor: Chunhui ZHANG, Beijing (CN)

(73) Assignee: BOE TECHNOLOGY GROUP CO., LTD., Beijing (CN)

(21) Appl. No.: 17/462,623

(22) Filed: Aug. 31, 2021

(30) Foreign Application Priority Data
Jan. 13, 2021 (CN) 202110040485.7

(51) Int. Cl. G16H 20/10; G16H 50/20; G16H 10/60

(52) U.S. Cl. CPC G16H 20/10; G16H 10/60; G16H 50/20

(57) ABSTRACT

Disclosed are a drug recommendation method, a drug recommendation apparatus, a drug recommendation system, an electronic device and a non-transitory storage medium. The drug recommendation method includes: obtaining patient information; determining, based on a drug knowledge graph and the patient information, a candidate drug set; scoring each drug in the candidate drug set and determining a target recommended drug based on a scoring result; and providing the target recommended drug.

### TECHNICAL FIELD

Embodiments of the present disclosure relate to a drug recommendation method, a drug recommendation apparatus, a drug recommendation system, an electronic device and a non-transitory storage medium.

### BACKGROUND

Deepening the reform of the medical and health system is a systematic project involving a wide range and great difficulty. On the one hand, the medical resources of our country are in short supply, which is difficult to meet the growing demand for medical services. On the other hand, various irrational drug use problems, such as drug abuse and irregular drug use, are more and more serious, which not only directly affect medical safety and quality, and bring health hazards to patients, but also cause huge economic losses to the society.

Meanwhile, with the progress of science and the development of the times, the explosion of knowledge poses severe challenges to the work of doctors, and the renewal and growth of knowledge in the medical field also exceed the learning and mastery limits of doctors. The diversity of drugs and the different pathological characteristics of patients complicate the drug treatments, and many factors affect the type and dosage of drugs. Therefore, in the case where an effective auxiliary medical decision-making method is absent, it is difficult to solve the current drug use problems by relying solely on the personal judgment of doctors, especially interns with little experience.

### SUMMARY

At least some embodiments of the present disclosure provide a drug recommendation method, which includes: obtaining patient information; determining, based on a drug knowledge graph and the patient information, a candidate drug set; scoring each drug in the candidate drug set and determining a target recommended drug based on a scoring result; and providing the target recommended drug.

For example, in the drug recommendation method provided by some embodiments of the present disclosure, the drug knowledge graph includes a single-drug knowledge graph; and the determining, based on the drug knowledge graph and the patient information, the candidate drug set, includes: determining, based on the single-drug knowledge graph and the patient information, the candidate drug set.

For example, in the drug recommendation method provided by some embodiments of the present disclosure, the scoring each drug in the candidate drug set and determining the target recommended drug based on the scoring result, includes: determining a disease of a patient based on the patient information; determining a matching degree score of each drug in the candidate drug set for the disease of the patient based on the single-drug knowledge graph, and sorting each drug in the candidate drug set base on the matching degree score; and taking a drug conforming to a predetermined sorting rule in the candidate drug set as the target recommended drug.

For example, in the drug recommendation method provided by some embodiments of the present disclosure, the single-drug knowledge graph includes a drug-indication-disease triple-tuple data set; and the determining the matching degree score of each drug in the candidate drug set for the disease of the patient based on the single-drug knowledge graph, includes: representing all of the drug-indication-disease triple-tuple data set in the single-drug knowledge graph as a bipartite graph; and performing a random walk in the bipartite graph based on a random walk algorithm, so as to calculate the matching degree score of each drug in the candidate drug set for the disease of the patient.

For example, in the drug recommendation method provided by some embodiments of the present disclosure, the bipartite graph includes a plurality of drug nodes corresponding to all drugs in the single-drug knowledge graph, a plurality of disease nodes corresponding to all diseases in the drug knowledge graph, and a path connecting any drug node and any disease node which have an indication relationship; and the performing the random walk in the bipartite graph based on the random walk algorithm, so as to calculate the matching degree score of each drug in the candidate drug set for the disease of the patient, includes: setting a random walk probability, and setting initial access probabilities of all nodes in the bipartite graph; in each walk process, taking a disease node corresponding to the disease of the patient as a starting point to start walking, and upon walking to any node, determining whether to continue to walk or stop the present walk process based on the random walk probability, and in case of stopping the present walk process, calculating access probabilities of all nodes in the bipartite graph based on an iterative formula as follows:

$$PR ( i ) = \{ \begin{matrix}α * {\sum }_{j ∈ i n ( i )} \frac{PR ( j )}{\vert out ( j ) \vert} & if ( i \neq D ) \\ ( 1 - α ) + α * {\sum }_{j ∈ i n ( i )} \frac{PR ( j )}{\vert out ( j ) \vert} & if ( i = D )\end{matrix} ,$$

where PR (i) represents an access probability of a node i, α represents the random walk probability, in(i) represents a set of all nodes pointing to the node i, a node j is any node in the in(i), and out(j) represents a set of all nodes pointed to the node j; and judging whether the above random walk process meets an iterative termination condition, if the iterative termination condition is not met, repeating the above random walk process, and if the iterative termination condition is met, stopping the above random walk process, and taking an access probability of a drug node corresponding to each drug in the candidate drug set as the matching degree score of the each drug in the candidate drug set for the disease of the patient.

For example, in the drug recommendation method provided by some embodiments of the present disclosure, the initial access probability of the disease node corresponding to the disease of the patient is set to 1, and the initial access probabilities of other nodes except the disease node corresponding to the disease of the patient is set to 0.

For example, in the drug recommendation method provided by some embodiments of the present disclosure, a value range of the random walk probability is [0.8, 0.9].

For example, in the drug recommendation method provided by some embodiments of the present disclosure, the single-drug knowledge graph includes a use-weight of a drug corresponding to each disease; and the determining the matching degree score of each drug in the candidate drug set for the disease of the patient based on the single-drug knowledge graph, includes: taking, based on the single-drug knowledge graph, a use-weight of each drug in the candidate drug set relative to the disease of the patient as the matching degree score of the each drug in the candidate drug set for the disease of the patient.

For example, the drug recommendation method provided by some embodiments of the present disclosure further includes: increasing, based on a selection condition of the target recommended drug, a use-weight of a selected target recommended drug relative to the disease of the patient in the single-drug knowledge graph.

For example, in the drug recommendation method provided by some embodiments of the present disclosure, the drug knowledge graph includes a drug combination knowledge graph; and the determining, based on the drug knowledge graph and the patient information, the candidate drug set, includes: determining, based on the drug combination knowledge graph and the patient information, the candidate drug set.

For example, in the drug recommendation method provided by some embodiments of the present disclosure, the patient information includes a disease of a patient; and the determining, based on the drug combination knowledge graph and the patient information, the candidate drug set, includes: inquiring all combined prescriptions having an indication relationship with the disease of the patient in the drug combination knowledge graph, so as to obtain the candidate drug set.

For example, the drug recommendation method provided by some embodiments of the present disclosure further includes: judging a drug combination necessity based on the patient information, and obtaining a judgment result of the drug combination necessity, wherein the judgment result of the drug combination necessity includes needing drug combination or not needing drug combination; wherein the drug knowledge graph includes a single-drug knowledge graph and a drug combination knowledge graph; the determining, based on the drug knowledge graph and the patient information, the candidate drug set, includes: in response to that the judgment result of the drug combination necessity is not needing drug combination, determining, based on the single-drug knowledge graph and the patient information, the candidate drug set, or, in response to that the judgment result of the drug combination necessity is needing drug combination, determining, based on the drug combination knowledge graph and the patient information, the candidate drug set.

For example, in the drug recommendation method provided by some embodiments of the present disclosure, the judging the drug combination necessity based on the patient information and obtaining the judgment result of the drug combination necessity, includes: judging, based on the patient information, the drug combination necessity by a binary classification model, so as to obtain the judgment result of the drug combination necessity.

For example, in the drug recommendation method provided by some embodiments of the present disclosure, the binary classification model is constructed by taking electronic medical records as training data, combining a medication guide, and adopting a decision tree algorithm.

For example, in the drug recommendation method provided by some embodiments of the present disclosure, the single-drug knowledge graph is constructed with a single drug as a core node.

For example, in the drug recommendation method provided by some embodiments of the present disclosure, the drug combination knowledge graph is constructed with a combined prescription as a core node, wherein the combined prescription includes at least two drugs.

For example, the drug recommendation method provided by some embodiments of the present disclosure further includes: performing, based on the drug knowledge graph and the patient information, a safety check on the target recommended drug, so as to obtain a check report of the target recommended drug.

For example, in the drug recommendation method provided by some embodiments of the present disclosure, the performing, based on the drug knowledge graph and the patient information, the safety check on the target recommended drug, so as to obtain the check report of the target recommended drug, includes: determining at least one selected from the group consisting of prohibition information, caution information and allergy information of the target recommended drug by inquiring the drug knowledge graph; and matching the at least one selected from the group consisting of the prohibition information, the caution information and the allergy information of the target recommended drug with the patient information, so as to obtain the check report of the target recommended drug, wherein in a case where the at least one selected from the group consisting of the prohibition information, the caution information and the allergy information of the target recommended drug successfully matches with the patient information, the check report of the target recommended drug includes at least a corresponding one selected from the group consisting of a prohibition reminder, a caution reminder and an allergy reminder.

For example, in the drug recommendation method provided by some embodiments of the present disclosure, the performing, based on the drug knowledge graph and the patient information, the safety check on the target recommended drug, so as to obtain the check report of the target recommended drug, further includes: in a case where the target recommended drug includes a drug combination, determining incompatibility information of various drugs in the drug combination by inquiring the drug knowledge graph, determining whether an incompatibility is existed between the various drug in the drug combination based on the incompatibility information of the various drugs in the drug combination, and providing an incompatibility reminder in response to that an incompatibility is existed between the various drugs in the drug combination, wherein the check report of the target recommended drug further includes the incompatibility reminder of the drug combination.

For example, in the drug recommendation method provided by some embodiments of the present disclosure, the performing, based on the drug knowledge graph and the patient information, the safety check on the target recommended drug, so as to obtain the check report of the target recommended drug, further includes: in a case where the target recommended drug includes a drug combination, traversing drug combined prescriptions appeared in electronic medical record big data, taking various drugs appeared in the drug combined prescriptions as nodes, setting initial edge-weights between various drugs to 0, and increasing, every time a combination of any two drugs appears in the electronic medical record big data, an edge-weight between the any two drugs, so as to form a network with weights; and determining, based on the network with weights, a safety of the drug combination by a graph search algorithm, wherein the check report of the target recommended drug further includes a safety determination result of the drug combination.

For example, the drug recommendation method provided by some embodiments of the present disclosure further includes: providing the check report of the target recommended drug, while providing the target recommended drug.

For example, in the drug recommendation method provided by some embodiments of the present disclosure, the providing the target recommended drug includes: providing a plurality of medication schemes, wherein each of the plurality of medication schemes includes at least one drug.

For example, in the drug recommendation method provided by some embodiments of the present disclosure, the patient information includes one or more of the following terms: a disease or disorder of a patient, a population attribute of the patient, concomitant/potential disease or disorder information of the patient, a diagnosis and treatment condition of the patient, job information of the patient, and a medication condition of the patient.

For example, the drug recommendation method provided by some embodiments of the present disclosure further includes: constructing the drug knowledge graph.

For example, the drug recommendation method provided by some embodiments of the present disclosure further includes: updating the drug knowledge graph based on a selection condition of the target recommended drug.

At least some embodiments of the present disclosure further provide a drug recommendation apparatus, which includes: a patient information interaction module, configured to obtain patient information; a candidate drug determination module, configured to determine a candidate drug set based on a drug knowledge graph and the patient information; a candidate drug scoring module, configured to score each drug in the candidate drug set, and to determine a target recommended drug based on a scoring result; and a user selection module, configured to provide the target recommended drug.

For example, the drug recommendation apparatus provided by some embodiments of the present disclosure further includes: a drug combination necessity judgment module, configured to judge a drug combination necessity based on the patient information, so as to obtain a judgment result of the drug combination necessity, wherein the judgment result of the drug combination necessity includes needing drug combination or not needing drug combination; wherein the drug knowledge graph includes a single-drug knowledge graph and a drug combination knowledge graph, and the candidate drug determination module being configured to determine the candidate drug set based on the drug knowledge graph and the patient information includes: the candidate drug determination module is configured to determine, in response to that the judgment result of the drug combination necessity is not needing drug combination, the candidate drug set based on the single-drug knowledge graph and the patient information, or to determine, in response to that the judgment result of the drug combination necessity is needing drug combination, the candidate drug set based on the drug combination knowledge graph and the patient information.

For example, the drug recommendation apparatus provided by some embodiments of the present disclosure further includes: a safety check module, configured to perform a safety check on the target recommended drug based on the drug knowledge graph and the patient information, so as to obtain a check report of the target recommended drug.

For example, the drug recommendation apparatus provided by some embodiments of the present disclosure further includes: a knowledge graph construction module, configured to construct the drug knowledge graph.

For example, in the drug recommendation apparatus provided by some embodiments of the present disclosure, the knowledge graph construction module is further configured to update the drug knowledge graph based on a selection condition of the target recommendation drug.

At least some embodiments of the present disclosure further provide a drug recommendation system, which includes a terminal and a drug recommendation apparatus; wherein the terminal is configured to send request data to the drug recommendation apparatus; and the drug recommendation apparatus is configured to: obtain patient information based on the request data; determine a candidate drug set based on a drug knowledge graph and the patient information; score each drug in the candidate drug set, and determine a target recommended drug based on a scoring result; and provide the target recommended drug to the terminal.

For example, the drug recommendation system provided by some embodiments of the present disclosure further includes: a physical examination system, configured to provide the patient information to the drug recommendation apparatus.

At least some embodiments of the present disclosure further provide an electronic device, which includes: a memory, configured to store computer readable instructions non-transitorily; and a processor, configured to execute the computer readable instructions, wherein upon the computer readable instructions being executed by the processor, the drug recommendation method provided by any one of the embodiments of the present disclosure is executed.

At least some embodiments of the present disclosure further provide anon-transitory storage medium, storing computer readable instructions non-transitorily, wherein upon the computer readable instructions being executed by a computer, the drug recommendation method provided by any one of the embodiments of the present disclosure is executed.

### BRIEF DESCRIPTION OF THE DRAWINGS

In order to clearly illustrate the technical solutions of the embodiments of the disclosure, the drawings of the embodiments will be briefly described in the following; it is obvious that the described drawings are only related to some embodiments of the disclosure and thus are not limitative to the disclosure.

FIG. 1 is a flowchart of a drug recommendation method provided by at least some embodiments of the present disclosure;

FIG. 2 is a flowchart of another drug recommendation method provided by at least some embodiments of the present disclosure;

FIG. 3 is a flowchart of further another drug recommendation method provided by at least some embodiments of the present disclosure;

FIG. 4 is an exemplary flowchart corresponding to step 300 shown in FIG. 1 provided by at least some embodiments of the present disclosure;

FIG. 5A is a schematic diagram of a drug-indication-disease triple-tuple data set provided by at least some embodiments of the present disclosure;

FIG. 5B is a bipartite graph constructed based on the drug-indication-disease triple-tuple data set shown in FIG. 5A;

FIG. 6 is a flowchart of still another drug recommendation method provided by at least some embodiments of the present disclosure;

FIG. 7 is an exemplary flowchart corresponding to step 390 shown in FIG. 6 provided by at least some embodiments of the present disclosure;

FIG. 8A is a schematic diagram of an interactive interface provided by at least some embodiments of the present disclosure;

FIG. 8B is a schematic diagram of another interactive interface provided by at least some embodiments of the present disclosure;

FIG. 9 is a schematic block diagram of a drug recommendation apparatus provided by at least some embodiments of the present disclosure;

FIG. 10A is a schematic block diagram of a drug recommendation system provided by at least some embodiments of the present disclosure;

FIG. 10B is a schematic block diagram of a terminal provided by at least some embodiments of the present disclosure;

FIG. 10C is a schematic block diagram of another drug recommendation system provided by at least some embodiments of the present disclosure;

FIG. 11 is a schematic block diagram of an electronic device provided by at least some embodiments of the present disclosure; and

FIG. 12 is a schematic diagram of a non-transitory storage medium provided by at least some embodiments of the present disclosure.

### DETAILED DESCRIPTION

In order to make objects, technical details and advantages of the embodiments of the disclosure apparent, the technical solutions of the embodiments will be described in a clearly and fully understandable way in connection with the drawings related to the embodiments of the disclosure. Apparently, the described embodiments are just a part but not all of the embodiments of the disclosure. Based on the described embodiments herein, those skilled in the art can obtain other embodiment(s), without any inventive work, which should be within the scope of the disclosure.

Unless otherwise defined, all the technical and scientific terms used herein have the same meanings as commonly understood by one of ordinary skill in the art to which the present disclosure belongs. The terms “first,” “second,” etc., which are used in the present disclosure, are not intended to indicate any sequence, amount or importance, but distinguish various components. Also, the terms “comprise,” “comprising,” “include,” “including,” etc., are intended to specify that the elements or the objects stated before these terms encompass the elements or the objects and equivalents thereof listed after these terms, but do not preclude the other elements or objects. The phrases “connect”, “connected”, etc., are not intended to define a physical connection or mechanical connection, but may include an electrical connection, directly or indirectly. “On,” “under,” “right,” “left” and the like are only used to indicate relative position relationship, and when the position of the object which is described is changed, the relative position relationship may be changed accordingly.

The present disclosure is described below with reference to some specific embodiments. In order to keep the following description of the embodiments of the present disclosure clear and concise, detailed descriptions of known functions and known components may be omitted. When any one component of an embodiment of the present disclosure appears in more than one of the accompanying drawings, the component is denoted by a same or similar reference numeral in each of the drawings.

With the development of computer technology and big data technology, building an auxiliary drug recommendation system based on a knowledge graph has become an important research direction. Current drug recommendation systems based on knowledge graph mostly generate a list of recommended drugs according to patient information and submit the list of recommended drugs to a patient or a doctor for choice, and the necessity of drug combination and the generation of a corresponding prescription are not considered. However, in clinical work, a reasonable drug combination can increase the efficacy of drugs and reduce the side effects of drugs, so there are more and more cases of drug combination. Because complexity and difficulty of drug combination are high, and the interaction between drugs is easy to cause adverse drug reactions, considering drug combination often increases the work difficulty of doctors. Therefore, an effective drug recommendation method is needed to assist doctors in medication work, so as to reduce the work burden of doctors.

At least some embodiments of the present disclosure provide a drug recommendation method. The drug recommendation method includes: obtaining patient information; determining, based on a drug knowledge graph and the patient information, a candidate drug set; scoring each drug in the candidate drug set and determining a target recommended drug based on a scoring result; and providing the target recommended drug.

At least some embodiments of the present disclosure further provide a drug recommendation apparatus, a drug recommendation system, an electronic device, and a non-transitory storage medium, which are corresponding to the drug recommendation method.

The drug recommendation method provided by the embodiments of the present disclosure can score and sort each drug in the candidate drug set to determine the target recommended drug, thereby effectively avoiding a huge recommendation results, facilitating a selection of a user, and reducing the probability of occurrence of medication side effects.

Hereinafter, some embodiments of the present disclosure and examples thereof will be described in detail with reference to the accompanying drawings. It should be understood that the specific embodiments described herein are only used to illustrate and explain the present disclosure and are not intended to limit the present disclosure.

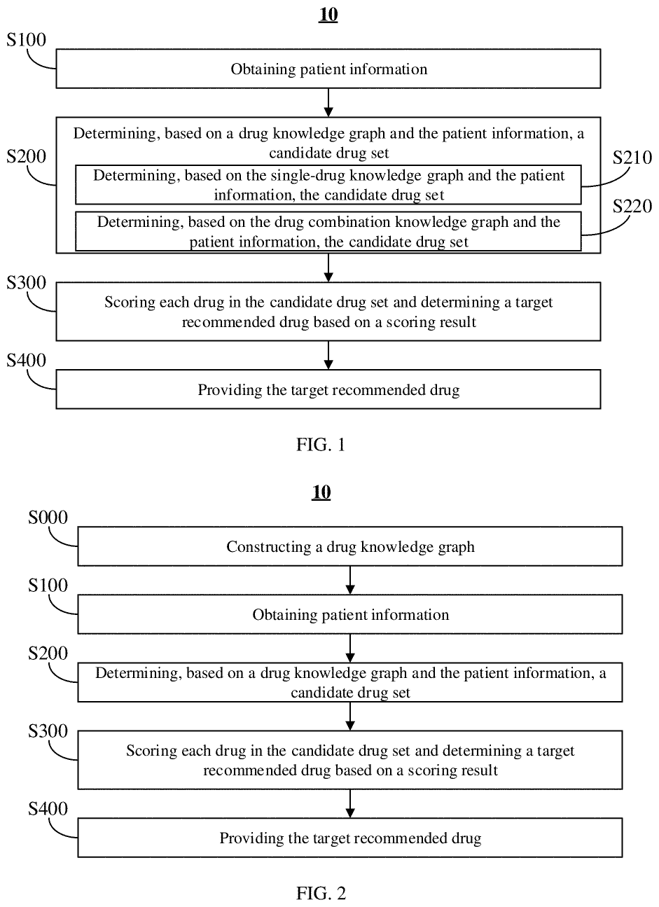

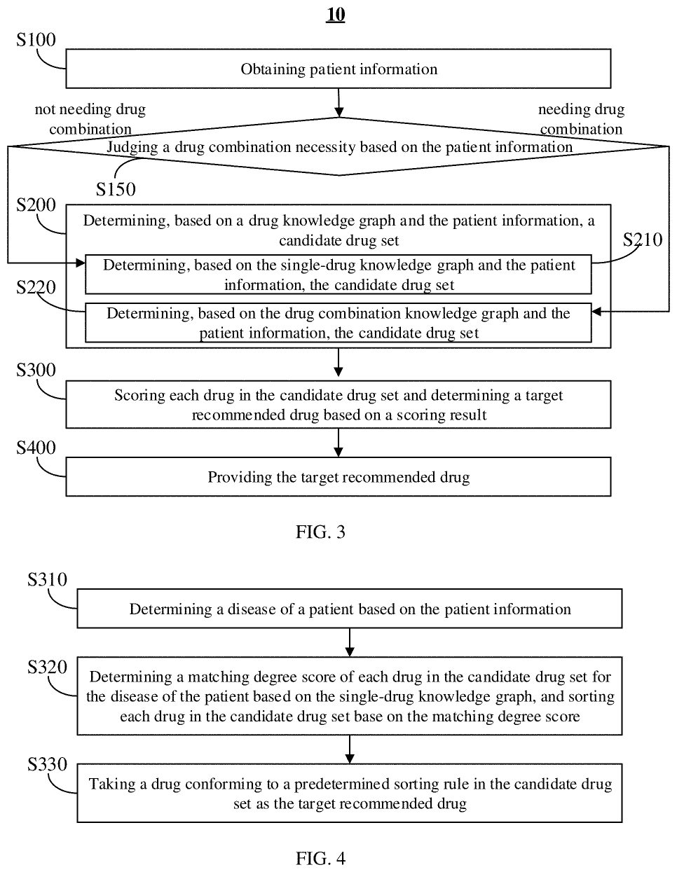

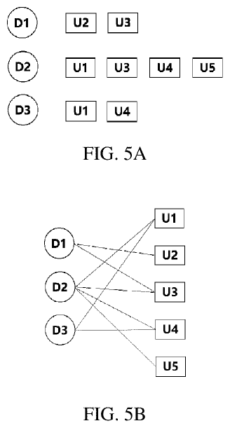

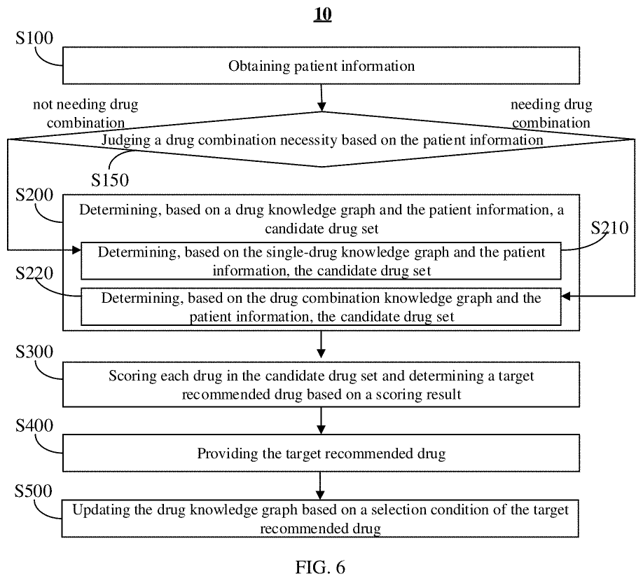

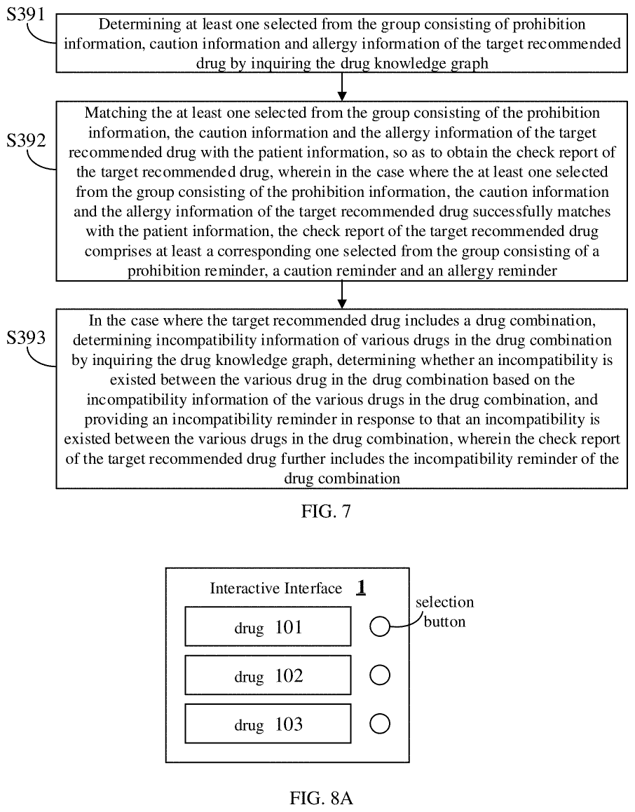

FIG. 1 is a flowchart of a drug recommendation method provided by at least some embodiments of the present disclosure; FIG. 2 is a flowchart of another drug recommendation method provided by at least some embodiments of the present disclosure; FIG. 3 is a flowchart of further another drug recommendation method provided by at least some embodiments of the present disclosure; FIG. 4 is an exemplary flowchart corresponding to step 300 shown in FIG. 1 provided by at least some embodiments of the present disclosure; FIG. 5A is a schematic diagram of a drug-indication-disease triple-tuple data set provided by at least some embodiments of the present disclosure; FIG. 5B is a bipartite graph constructed based on the drug-indication-disease triple-tuple data set shown in FIG. 5A; FIG. 6 is a flowchart of still another drug recommendation method provided by at least some embodiments of the present disclosure; and FIG. 7 is an exemplary flowchart corresponding to step 390 shown in FIG. 6 provided by at least some embodiments of the present disclosure.

For example, the drug recommendation method provided by the embodiments of the present disclosure can be applied to scenarios such as a doctor issuing a prescription based on patient information and a patient using an application (e.g., APP, etc.) to obtain a recommended prescription, etc. For example, the drug recommendation method provided by the embodiments of the present disclosure can be performed by a computing device, and the computing device includes any electronic device with computing function, such as a smart phone, a notebook computer, a tablet computer, a desktop computer, a server, etc., without being limited in the embodiments of the present disclosure. For example, the computing device has a central processing unit (CPU) or a graphics processing unit (GPU), and the computing device further includes a memory. The memory is, for example, a non- transitory memory (e.g., Read Only Memory (ROM)), and codes of an operating system are stored in the memory. For example, the memory further stores codes or instructions, and the drug recommendation method provided by the embodiments of the present disclosure can be implemented by running these codes or instructions.

For example, in at least some embodiments of the present disclosure, as shown in FIG. 1, the drug recommendation method 10 can include the following steps S100 to S400.

Step S100: obtaining patient information.

For example, in some embodiments, the patient information obtained in step S100 can generally include one or more of the following terms: (1) a disease or disorder of a patient, for example, including a consultation disease, a consultation symptom, and a pulse condition, etc.; (2) a population attribute of the patient, including age, gender, height, weight, physiological stage (pregnancy, menstruation, etc.), etc.; (3) concomitant/potential disease or disorder information of the patient, including a concomitant disease, a concomitant symptom, a personal medical history, a family medical history, etc.; (4) a diagnosis and treatment condition of the patient, including diagnosis and treatment information, such as a current/past surgery, an current/past examination, an current/past inspection, a current/past radiotherapy, a current/past chemotherapy, etc.; (5) job information of the patient, such as an athlete, a driver, a machine operator, etc.; (6) a medication condition of the patient, including drug allergy history, past medication and effect, etc.

For example, in some embodiments, the patient information described above can be entered into the computing device by a user (e.g., a doctor). For example, in some other embodiments, the patient information described above can be extracted from a physical examination report and/or an electronic medical record of the patient by a computing device using a related technology in the field of natural language processing (e.g., an optical character recognition technology, etc.). For example, in some examples, abnormal indexes can be extracted from the medical examination report of the patient, and information, such as the disease or disorder of the patient, the concomitant/potential disease or disorder information of the patient, etc., can be determined based on the abnormal indexes. For example, in some other embodiments, a human-computer interactive interface can be set, a number of pre-set questions (these questions are mainly used to obtain patient information) can be answered by the patient, and then the patient information can be extracted based on the question-and-answer record of the patients. Of course, the patient information can also be directly inputted by the patient. It should be noted that the manner of obtaining the patient information is not limited in the embodiments of the present disclosure.

For example, in some embodiments, in step S100, various labels can be extracted as patient features based on the patient information by means of named entity recognition or the like, so as to obtain structured patient information (i.e., the patient features). For example, in some examples, a statistical-based machine learning algorithm, such as conditional random field (CRF) model, maximum entropy Markov model (MEMM), etc., can be used to perform the named entity recognition to extract labels; for example, in some other examples, a deep learning algorithm, such as long-short term memory (LSTM) model or bi-directional long-short term memory (Bi-LSTM) model, etc., can be used to perform the named entity recognition to extract labels; for example, in some other examples, the aforementioned algorithms can further be combined (e.g., combining the CRF model and the Bi-LSTM model, etc.) to perform the named entity recognition, so as to extract labels; and the embodiments of the present disclosure are not limited to these cases.

For example, in some embodiments, in step S100, corresponding to the patient information, the patient features generally includes the following six categories: the disease or disorder of the patient, the population attribute of the patient, the concomitant/potential disease or disorder information of the patient, the diagnosis and treatment condition of the patient, the job information of the patient, and the medication condition of the patient. It should be understood that one part of the patient features can be directly read from the patient information, and the other part of the patient features need to be obtained by extracting labels from the patient information. For example, in some examples, the disease or disorder of the patient (e.g., consultation disease, consultation symptom, etc.) can be directly read, so as to obtain the labels of the disease or disorder of the patient (e.g., the specific type of consultation disease, the specific type of consultation symptom, etc.), directly. For example, in some examples, according to age of the patients, patients can be divided into several categories, including newborns (people within 28 days of birth), infants (under 1 year old), young children (1-3 years old), children (under 14 years old), and adolescents (14-18 years old), young people (14-35 years old), middle-aged and elderly people (45-60 years old), aged people (over 60 years old), etc., so that the patient can be directly associated with a corresponding age label according to a rule. For example, in some examples, a first-level label can be determined according to the drug allergy history of the patient. For example, “having drug allergy history” and “having no drug allergy history” can be used as the first-level labels. Further, in the case where the patient has drug allergy history, a second-level label can further be determined, and for example, “penicillin allergy” and “cephalosporin allergy” can be used as the second-level labels. For example, in some examples, the first-level label of disease of, for example, a patient with hypertension, can be directly read, such as “hypertension”; and in this case, a second-level label can be further determined according to the specific values of diastolic and systolic blood pressure, such as “primary hypertension”, “secondary hypertension”, “tertiary hypertension”, etc. It should be understood that the embodiments of the present disclosure do not limit the specific rule of user labels, which can be set according to actual requirements. It should be understood that the purpose of extracting patient features in step S100 is mainly to structure and standardize the patient information, so as to be used for related operations in subsequent steps.

For example, the patient features extracted in step S100 can be used as the patient information actually used for related operations in subsequent steps. It should be understood that the patient features used in different subsequent steps are not necessarily the same, that is, the patient features extracted in step S100 can be selectively used in different subsequent steps as needed. It should be noted that the patient features are not limited to the features listed above, but may also include other features, which can be determined according to actual needs, without being limited in the embodiments of the present disclosure.

Step S200: determining, based on a drug knowledge graph and the patient information, a candidate drug set.

For example, in some embodiments, the drug knowledge graph in the step S200 includes at least one of a single-drug knowledge graph and a drug combination knowledge graph.

For example, in some embodiments, the natural language processing (NLP) technology can be used for extracting drug information and medication information included in medical texts such as drug instructions and medication guides, and further forming a structured knowledge graph (e.g., the single-drug knowledge graph and the drug combination knowledge graph described above), so as to support an intelligent drug recommendation function.

For example, in some embodiments, the single-drug knowledge graph can be constructed with a single drug as a core node, that is, the single-drug knowledge graph is constructed around single drug. For example, in some embodiments, the natural language processing technologies, such as named entity recognition, relationship extraction, entity alignment, etc., can be used for extraction on drug instructions, etc., so as to obtain a single-drug knowledge graph including a plurality of entities and a plurality of relationships, such as general drug name, drug commodity name, drug composition, indications, allergy information, prohibited population, cautious population, prohibited disease, cautious disease, prohibited medical history, cautious medical history, prohibited symptom, cautious symptom, prohibited diagnosis and treatment, and cautious diagnosis and treatment. For example, the algorithms, such as the CRF model, the MEMM model, the LSTM model, the Bi-LSTM model, etc., can be used for performing the named entity recognition. For example, a rule-based method (e.g., based on trigger words/strings, based on dependency syntax), a method based on supervised learning (e.g., machine learning, deep learning), a method based on semi-supervised/unsupervised learning (e.g., based on bootstrapping) or the like, can be used for performing the relationship extraction. For example, the semantic similarity algorithm described below can be used for performing the entity alignment.

For example, for the single drug “heat-clearing and detoxicating oral liquid”, the knowledge covering a plurality of relationships and entities such as “drug composition”, “indications”, “prohibited population”, “cautious population”, “allergy information”, etc., can be extracted from the texts of the drug instruction through the natural language processing technology, so as to obtain the corresponding content of a knowledge graph. Similarly, for the single drug “antiviral oral liquid”, the corresponding content of a knowledge graph can also be obtained. And for example, the indications of the heat-clearing and detoxicating oral liquid and the indications of the antiviral oral liquid both include “influenza”. Therefore, an indirect connection relationship between the drug entities of these two single drugs can be established through the disease entity “influenza”.

For example, in some embodiments, combined prescriptions can be collated and extracted from a medical professional book, such as a medical guide, a medication guide etc., and from an electronic medical record, etc.; a combined prescription including at least two drugs is taken as a new drug, which is taken as a core node to construct a drug combination knowledge graph, that is, the drug combination knowledge graph is constructed around combined prescription. For example, in some embodiments, the drug combination knowledge graph includes a plurality of entities and a plurality of relationships, such as single drugs being contained, indications, applicable population, and medication method. For example, treating a combined prescription that includes at least two drugs as a new drug can effectively improve the accuracy of the information in the knowledge graph.

For example, similar to the single-drug knowledge graph, it can be known, from professional medical data such as the medical guide and the medication guide, etc., that the drug “valsartan” and the drug “hydrochlorothiazide” are usually used in combination (that is, as a combined prescription) to treat moderate hypertension. When constructing a drug combination knowledge graph, the above two drugs are taken as a node “valsartan+hydrochlorothiazide”, and the knowledge of relationships and entities including “single drugs being contained”, “indications” and “applicable population”, etc., is extracted from the above professional medical data through natural language processing technology, so as to obtain the corresponding content of a knowledge graph.

For example, drug combination (and combined prescription) is a medication method in which two or more drugs are applied simultaneously or successively for the treatment of a certain disease, and the purpose of drug combination is mainly to improve the efficacy and/or reduce the toxic or side effects of the drugs, etc. For example, the above-mentioned combined prescription consisting of valsartan and hydrochlorothiazide can be used for treating moderate hypertension; the two drugs have a synergistic effect, valsartan can cause a slight increase in blood potassium, while hydrochlorothiazide can cause a decrease in blood potassium, and the combination of them can offset the adverse reactions of each other. In the embodiments of the present disclosure, the combined prescription belongs to one form of drug union, and the other form of drug union is that various single drugs included therein are respectively used to treat different diseases of the patient (this form is different from the combined prescription).

For example, the drug combination can also be a drug union, which is not limited here.

For example, in some embodiments, as shown in FIG. 2, the drug recommendation method 10 can further include step S000: constructing a drug knowledge graph. Therefore, the drug knowledge graph constructed in step S000 can be used for related operations in the subsequent step S200.

For example, in some embodiments, the drug knowledge graph can be constructed in advance, and the drug knowledge graph constructed in advance can be stored in a local terminal or a remote server in advance; and in this case, the drug recommendation method may omit step S000. For example, in some other embodiments, the drug knowledge graph can also be constructed when the drug recommendation method 10 is implemented (e.g., constructed by a server), and in this case, the drug recommendation method can include step S000. Of course, the drug knowledge graph can also be read from other devices, which is not specifically limited in the embodiments of the present disclosure.

For example, in some embodiments, as shown in FIG. 1, in the case where the drug knowledge graph includes a single-drug knowledge graph, step S200 can include step S210: determining, based on the single-drug knowledge graph and the patient information, the candidate drug set.

For example, in some embodiments, a knowledge graph query and search algorithm can be used to determine a candidate drug for symptomatic treatment from the single-drug knowledge graph based on the aforementioned patient features. For example, in some embodiments, the candidate drug set usually includes a plurality of candidate drugs, and the embodiments of the present disclosure include but are not limited thereto. It should be understood that the patient features used in step S210 are consistent with at least part of the patient features covered by the single-drug knowledge graph. For example, in some embodiments, the patient features used in step S210 can include the disease or disorder of the patient, etc. The embodiments of the present disclosure include but are not limited thereto. It should be understood that, in the embodiments of the present disclosure, each candidate drug in the candidate drug set serves as a medication scheme in the case where a single-drug knowledge graph is used to determine the candidate drug set.

For example, all drugs having an indication relationship with the disease of the patient can be inquired in the single-drug knowledge graph, so as to obtain a candidate drug set. For example, the disease or disorder information of the patient can be linked to a correct target entity (i.e., the disease entity corresponding to the disease or disorder of the patient) in the single-drug knowledge graph through entity linking algorithm. If a correct target entity can be linked, the candidate drug set can be obtained by inquiring all drug entities having an “indication” relationship with the target entity in the single-drug knowledge graph. If a correct target entity cannot be linked, the current single-drug knowledge graph has not yet included this disease or disorder, and in this case, the single-drug knowledge graph can be updated according to the needs (that is, a corresponding disease entity and various entities and relationships related to the disease entity are supplemented). Taking that the disease of the patient is hypertension as an example, the hypertension is linked to a correct target entity (i.e., the disease entity “hypertension”) in the knowledge graph through the entity linking algorithm, and then the single-drug knowledge graph is inquired to obtain all drug entities having an “indication” relationship with the target entity “hypertension”, so that the candidate drug set is obtained.

It should be understood that due to the wide variety of drugs, the candidate drug set generated in step S210 is usually relatively large (that is, a large number of candidate drugs are included), thereby not facilitating the user to make a choice. Therefore, in the embodiments of the present disclosure, the candidate drug set generated in step S210 can be optimized and filtered through step S300, so that the user can make a selection more conveniently.

For example, in some embodiments, as shown in FIG. 1, in the case where the drug knowledge graph includes a drug combination knowledge graph, step S200 can include step S220: determining, based on the drug combination knowledge graph and the patient information, the candidate drug set.

For example, in some embodiments, a knowledge graph query and search algorithm can be used to determine a candidate combined prescription from the drug combination knowledge graph based on the aforementioned patient features. For example, in some embodiments, the candidate drug set usually includes one or a plurality of candidate combined prescriptions, and the embodiments of the present disclosure include but are not limited thereto. It should be understood that the patient features used in step S220 is consistent with at least part of patient features covered by the drug combination knowledge graph. For example, in some embodiments, the patient features used in the step S220 can include the disease or disorder of the patient and the population attribute of the patient, etc. The embodiments of the present disclosure include but are not limited thereto. It should be understood that, in the embodiments of the present disclosure, each candidate combined prescription in the candidate drug set serves as a medication scheme in the case where a drug combination knowledge graph is used to determine the candidate drug set.

For example, all combined prescriptions having an indication relationship with the disease of the patient can be inquired in the drug combination knowledge graph, so as to obtain a candidate drug set. For example, the disease or disorder information of the patient can be linked to a correct target entity (i.e., the disease entity corresponding to the disease or disorder of the patient) in the drug combination knowledge graph through entity linking algorithm. If a correct target entity can be linked, the candidate drug set can be obtained by inquiring all drug entities having an “indication” relationship with the target entity (a drug entity in the drug combination knowledge graph is a combined prescription) in the drug combination knowledge graph. If a correct target entity cannot be linked, the current drug combination knowledge graph has not yet included this disease or disorder, and in this case, the drug combination knowledge graph can be updated according to the needs (that is, a corresponding disease entity and various entities and relationships related to the disease entity are supplemented). Taking that the disease of the patient is moderate hypertension as an example, the moderate hypertension is linked to a correct target entity (i.e., the disease entity “moderate hypertension”) in the knowledge graph through the entity linking algorithm, and then the knowledge graph is inquired to obtain all combined prescription entities having an “indication” relationship with the target entity “moderate hypertension”, so that the candidate drug set is obtained.

It should be understood that combined prescriptions are usually less optional, so that all combined prescriptions obtained by the query and search can be added to the candidate drug set without increasing the burden of user selection. Therefore, in the embodiments of the present disclosure, the candidate drug set generated in step S220 is allowed to be directly provided to the user for selection, that is, in this case, the related operation in step S300 can be omitted.

For example, in some embodiments, as shown in FIG. 3, before step S200, the drug recommendation method can further include step S150: judging a drug combination necessity based on the patient information, and obtaining a judgment result of the drug combination necessity, wherein the judgment result of the drug combination necessity includes needing drug combination or not needing drug combination. Therefore, step S200 can include: in response to that the judgment result of the drug combination necessity is not needing drug combination, determining, based on the single-drug knowledge graph and the patient information, the candidate drug set (that is, the related operation in step S210 is performed), or, in response to that the judgment result of the drug combination necessity is needing drug combination, determining, based on the drug combination knowledge graph and the patient information, the candidate drug set (that is, the related operation in step S220 is performed).

For example, in some embodiments, a binary classification model can be used for judging the drug combination necessity based on the patient information, so as to obtain a judgment result of the drug combination necessity. For example, in some embodiments, the binary classification model can be constructed by taking electronic medical records as training data, combining a medication guide, and adopting a decision tree algorithm. For example, in some embodiments, when constructing the binary classification model, the patient features being considered include the population attribute of the patient, the consultation disease and the consultation symptom in the disease of the patient, the concomitant disease, the concomitant symptom and the personal medical history in the concomitant/potential disease or disorder information of the patient, the allergy history and the past medication and effect in the medication condition of the patient, etc.; correspondingly, these patient features also need to be used when judging the drug combination necessity in step S150.

For example, in some embodiments, electronic medical record data can be used to perform the decision tree construction, and expert knowledge such as a medication guide and the like can be used to test the decision tree classification model (i.e., the aforementioned binary classification model). For example, in the case where the amount of training data (i.e., the electronic medical record data) is small or the distribution thereof is uneven, the decision tree classification model obtained by training may violate expert knowledge such as the medication guide and the like; and in this case, the training data and the construction process of the decision tree need to be adjusted, and the training is performed again. For example, the specific process of the training is as follows:

First, performing data annotation on the electronic medical record to mark out the patient feature information in the electronic medical record, such as the population attribute of the patient, the consultation disease, the consultation symptom, the concomitant disease, the concomitant symptom, the medical history, the allergy history, etc., and whether drug combination is used for the patient in the medical record. Then, using the above-mentioned annotated data to construct a decision tree classification model: (1) constructing a root node, wherein all training data is regarded as a root node; (2) selecting an optimal feature, wherein each classification feature is traversed, and a feature which can better classify the training data is selected as the optimal feature; (3) generating a decision tree, wherein step (2) is repeated until all the data is completely classified. Finally, a constructed decision tree classification model can be obtained.

For example, in the construction step (2) of the decision tree classification model described above, the optimal feature is a certain classification feature at the current node, and the data can be classified best through the classification feature. For example, in the embodiments of the present disclosure, the classification features can include the patient feature information, such as the population attribute of the patient, the consultation disease, the consultation symptom, the concomitant disease, the concomitant symptom, the medical history, the allergy history, etc. For example, according to the difference between decision tree classification models, the evaluation index for the best classification may also be different. For example, commonly used decision tree classification models include ID3 model, C4.5 model, CART model, etc., and each of the above models has a corresponding evaluation index (also called “feature selection index”). Taking the CART model as an example, the corresponding feature evaluation index of the CART model is the Gini index. The Gini index reflects the weighted sum of the purity of information in each category after using a certain classification feature a for classification. The smaller the Gini index, the higher the purity, and the better the classification effect of this feature. For example, the Gini index of the classification feature a is defined as follows:

$$Gini\_index ( D , a ) = {\sum }_{v = 1}^{V} \frac{\vert {D}^{v} \vert}{\vert D \vert} Gini ( {D}^{v} )$$

where D represents the data set to be classified, Gini_index(D, a) represents the Gini index of the classification feature a, V represents the number of all values of the classification feature a. the decision tree will have V branches after the classification feature a, D^{v} represents the data set of the v-th branch, Gini (D^{v}) represents the Gini value of the data set D^{v}. The Gini value reflects the probability that category labels of two samples which are randomly selected from the data set are inconsistent, and the smaller the Gini value, the higher the purity of the data set. For example, in the embodiments of the present disclosure, the category labels include two categories, i.e., needing drug combination and not needing drug combination. For example, the calculation formula of the Gini value is as follows:

$$Gini ( {D}^{v} ) = 1 - {\sum }_{k = 1}^{K} {p}_{k}^{2}$$

where D^{v} represents a data set, K represents the number of categories in the data set (in the embodiments of the present disclosure, K=2), and Pk represents the proportion of the k-th category in the data set.

For example, in some embodiments, firstly, data annotation can be performed on the electronic medical record to mark out the patient feature information in the electronic medical record, such as the population attribute of the patient, the consultation disease, the consultation symptom, the concomitant disease, the concomitant symptom, the medical history, the allergy history, etc., and whether drug combination is used for the patient in the medical record. Then, the above-mentioned annotation data can be used to construct the decision tree classification model: (1) constructing a root node, wherein all training data is regarded as a root node; (2) selecting an optimal feature, wherein each classification feature is traversed based on the Gini index, the Gini index is calculated after classification using each feature, and the feature which minimizes the Gini index, namely, the feature which achieves the optimal classification of the data, is selected as the optimal feature; assuming that current features include age, blood pressure, etc., the feature of age includes two values, greater than 60 years old and less than 60 years old, and the feature of blood pressure includes four values, normal blood pressure, mild hypertension, moderate hypertension and severe hypertension, and assuming that the calculated Gini index is G1 after the data set is classified by using the feature of age, while the calculated Gini index is G2 after the data set is classified by using the feature of blood pressure, and then, the feature with the smaller Gini index is selected as the classification feature of the current node; (3) iterating the above step to generate a decision tree, wherein step (2) is repeated until all the data is completely classified. Finally, a constructed decision tree classification model can be obtained.

For example, after the decision tree classification model is trained, when the patient features information, such as the population attribute of the patient, the consultation disease, the consultation symptom, the concomitant disease, the concomitant symptom, the medical history, the allergy history, etc., is inputted into the decision tree classification model, and the classification result of the patient, that is, whether drug combination is needed or not, is outputted by the model.

It should be noted that in the process of judging the drug combination necessity in step S150, only binary classification is needed to be performed, so that the accuracy and feasibility of the drug recommendation can be effectively improved. By judging the drug combination necessity, the candidate drug set subsequently generated can be more targeted to the disease of the patient, and the rationality and effectiveness of the drug recommendation can be improved.

Step S300: scoring each drug in the candidate drug set and determining a target recommended drug based on a scoring result.

For example, in some embodiments, in the case where the drug knowledge graph includes a single-drug knowledge graph, the relevant operation in step S300 can be performed according to the single-drug knowledge graph. For example, in some embodiments, a scoring function can be designed based on a graphics algorithm and in combination with the characteristics of drugs themselves, and the drugs in the candidate drug set can be sorted. For example, in some embodiments, as shown in FIG. 4, step S300 can include the following steps S310 to S330.

Step S310: determining a disease of a patient based on the patient information.

For example, in some embodiments, in step S310, the disease of the patient can be determined according to the disease or disorder of the patient in the patient features described above.

Step S320: determining a matching degree score of each drug in the candidate drug set for the disease of the patient based on the single-drug knowledge graph, and sorting each drug in the candidate drug set base on the matching degree score.

For example, in some embodiments, the single-drug knowledge graph can include a drug-indication-disease triple-tuple data set. In this case, the “determining a matching degree score of each drug in the candidate drug set for the disease of the patient based on the single-drug knowledge graph” in step S320 can include the following steps S321 and S322.

Step S321: representing all of the drug-indication-disease triple-tuple data set in the single-drug knowledge graph as a bipartite graph.

For example, in some embodiments, in step S321, all of the disease entities D (disease) and drug entities U (drug) in the single-drug knowledge graph can be taken as nodes, and the indication relationship between a disease entity D (disease) and a drug entity (drug) U (drug) can be taken as an edge, thus constructing a bipartite graph. That is, the bipartite graph includes a plurality of drug nodes corresponding to all drugs in the single-drug knowledge graph, a plurality of disease nodes corresponding to all diseases in the drug knowledge graph, a path connecting any drug node and any disease node which have an indication relationship.

For example, illustratively, as shown in FIG. 5A, the current drug-indication-disease triple-tuple data set includes disease entities D=[D1, D2, D3] and drug entities U=[U1, U2, U3, U4, U5], and a disease entity and a drug entity located in a same row have an indication relationship, that is, the drug located in a certain row can be used for treating the disease located in the same row. For example, as shown in FIG. 5A, the drugs U2 and U3 can be used for treating the disease D1, the drugs U1, U3, U4, and U5 can be used for treating the disease D2, and the drugs U1 and U4 can be used for treating the disease D3. The drug-indication-disease triple-tuple data set shown in FIG. 5A can be represented as a bipartite graph G(V, E) shown in FIG. 5B. For example, V is the vertex set formed of disease entities D and drug entities U, namely [D1, D2, D3, U1, U2, U3, U4, U5], and E represents a corresponding edge e (D, U) between each binary-tuple (D, U). If an indication relationship is existed between the binary-tuple (D, U), E=1 (that is, a solid line connection is existed between the binary-tuple, as shown in FIG. 5B), and if an indication relationship is not existed between the binary-tuple (D, U), E=0 (that is, a solid line connection is not existed between the binary-tuple, as shown in FIG. 5B).

Step S322: performing a random walk in the bipartite graph based on a random walk algorithm, so as to calculate the matching degree score of each drug in the candidate drug set for the disease of the patient.

For example, in practical applications, the more indications a drug is oriented to, the greater the probability that it will produce side effects, and the more serious the side effects may be. Based on the characteristics of the drug itself, the embodiments of the present disclosure have the following definitions for having a high matching degree score between a drug entity and a disease entity: (1) the length of the path connecting two vertices (i.e., the vertex of the drug entity and the vertex of the disease entity) is relatively short; (2) the two vertices are connected through many paths; (3) the path connecting the two vertices does not pass through a vertex with a large out-degree (that is, the number of paths directly derived from the vertex is large). For example, considering the above-mentioned conditions (1)-(3) comprehensively, the higher the degree of compliance with the above-mentioned conditions (1)-(3), the higher the matching degree score.

For example, illustratively, in the embodiment shown in FIG. 5A and FIG. 5B, assuming that the disease of the patient is D2, the plurality of candidate drugs in the candidate drug set include U1, U3, U4, U5; in this case, in step S322, the matching degree scores of the plurality of candidate drugs U1, U3, U4, U5 for the disease D2 of the patient can be calculated through the algorithm based on random walk, and then the plurality of candidate drugs U1, U3, U4, U5 can be sorted from high to low according to the matching degree scores. For example, in some embodiments, step S322 can include the following steps S322A to S322C.

Step S322A: setting a random walk probability, and setting initial access probabilities of all nodes in the bipartite graph.

For example, the initial access probability of the disease node corresponding to the disease of the patient is set to 1, and the initial access probabilities of other nodes except the disease node corresponding to the disease of the patient is set to 0.

Step S322B: in each walk process, taking a disease node corresponding to the disease of the patient as a starting point to start walking, and upon walking to any node, determining whether to continue to walk or stop the present walk process based on the random walk probability, and in case of stopping the present walk process, calculating access probabilities of all nodes in the bipartite graph based on an iterative formula as follows:

$$PR ( i ) = \{ \begin{matrix}α * {\sum }_{j ∈ i n ( i )} \frac{PR ( j )}{\vert out ( j ) \vert} & if ( i \neq D ) \\ ( 1 - α ) + α * {\sum }_{j ∈ i n ( i )} \frac{PR ( j )}{\vert out ( j ) \vert} & if ( i = D )\end{matrix}$$

where PR(i) represents an access probability of the node i, a represents the random walk probability, in(i) represents a set of all nodes pointing to the node i, a node j is any node in the in(i), and out(j) represents a set of all nodes pointed to the node j.

For example, the above formula is used for calculating the probability that the node i is accessed after each walk. The upper formula in the curly bracket on the right side of the equal sign indicates the access probability of any node i other than the disease node D corresponding to the disease of the patient (according to the aforementioned assumption, D=D2); that is, the sum of, the probability PR(j) that every node j pointing to the node i is accessed multiplied by the probability a of continuing to walk and then divided by the number of all nodes connected to the node j, is the access probability of the node i. The lower formula in the curly bracket on the right side of the equal sign indicates the access probability of the disease node D (according to the aforementioned hypothesis, D=D2) corresponding to the disease of the patient, in which the probability (1-α) of stopping the present walk process is further provided in addition to the above probability. For example, the random walk probability a can be set according to actual needs. For example, in some embodiments, the value range of the random walk probability is [0.8, 0.9].

Step S322C: judging whether the above random walk process meets an iterative termination condition; if the iterative termination condition is not met, repeating the above random walk process; and if the iterative termination condition is met, stopping the above random walk process, and taking an access probability of a drug node corresponding to each drug in the candidate drug set as the matching degree score of the each drug in the candidate drug set for the disease of the patient.

For example, in some examples, the iteration termination condition described above is that the access probability of each node is basically unchanged or the change thereof is less than a certain threshold after a plurality of random walk processes, that is, the access probability of each node is converged. For example, in some other examples, the iteration termination condition described above is that the number of random walk processes or the number of random walk steps reaches a predetermined number. The embodiments of the present disclosure are not limit to these cases.

Hereinafter, the algorithm based on random walk is described by taking the embodiment shown in FIG. 5A and FIG. 5B as an example, and it is assumed that the disease of patient is D2.

For example, for the embodiment shown in FIG. 5A and FIG. 5B, the walk can be start from the node (i.e., the vertex) D2 corresponding to the disease of the patient (that is, the node D2 corresponding to the disease of the patient is taken as the starting node of the random walk); when reaching any node, the walk may be stopped with the probability of 1-α and the walk is restarted from D2, or the walk is continued with the probability of a (0<α<1) and a node is randomly selected from the nodes pointed to the current node according to a uniform distribution to walk down. After many rounds of walking, the probability that each vertex is accessed (i.e., the access probability which is used for characterizing the matching degree score) will converge and become stable, so that the sorting can be performed according to the access probability described above.

For example, before executing the random walk algorithm, the initial access probability value of each node needs to be initialized. For example, in the case where recommendation needs to be performed with respect to the node D2 corresponding to the disease of the patient, the initial access probability of the node D2 can be set to 1, the initial access probabilities of other nodes can be set to 0, and then the above iterative formula can be used for calculating.

For example, in some examples of the embodiment shown in FIG. 5A and FIG. 5B, a is set to 0.85, the starting node of each random walk process is always the node D2, the initial value of PR(D2) is 1, the initial value of PR(i) is 0 (i≠D2), and the maximum number of walking steps is set to 100. In these 100 walking steps, each walk is an iteration. According to the above iterative formula, after each walk, the access probability values (i.e., the PR values) of all nodes in the bipartite graph can be calculated. After 100 iterations, all nodes can obtain a final PR value. For example, in a specific example, after 100 iterations according to the above conditions, the obtained result is [D1: 0.086, D2: 0.269, D3: 0.114, U1: 0.186, U2: 0.074, U3: 0.152, U4: 0.196, U5: 0.167], where the value of each node is the PR value of the node. For example, the PR values of the nodes U1, U3, U4, and U5 can be taken as the matching degree scores of the candidate drugs U1, U3, U4, and U5 for the disease D2 of the patient, respectively, so that the plurality of candidate drugs U1, U3, U4, U5 can be sorted according to the magnitudes of the matching degree scores, and the sorting order thereof is U4, U1, U5, U3.

It should be understood that the embodiment shown in FIG. 5A and FIG. 5B is illustrative. In practical applications, all the drug-indication-disease triple-tuple data sets in the drug knowledge graph are much more than the drug-indication-disease triple-tuple data set shown in FIG. 5A and FIG. 5B, but the principle of scoring and sorting is basically the same. It should also be understood that the numerical value of a and the numerical value of the number of iterations (i.e., the maximum number of walking steps) are also illustrative, and both of them can be set according to actual needs. It should be noted that the setting of the number of iterations usually needs to take into account the calculation accuracy and the time complexity of the calculation. For example, if the number of iterations is too large, the time complexity of the calculation is often be increased; and if the number of iterations is too small, the calculation accuracy is often be reduced. In practical applications, the number of iterations is usually set to a moderate value (this value can be determined according to actual needs), that is, while reducing the time complexity of the calculation, losing a certain amount of calculation accuracy is allowed (as long as the sorting result is not affected). For example, in some other embodiments, the single-drug knowledge graph can be used for recording the medication habits of each disease, so that the single-drug knowledge graph can include the use-weight (e.g., the number of uses) of any drug corresponding to each disease. In this case, the “determining the matching degree score of each drug in the candidate drug set for the disease of the patient based on the single-drug knowledge graph” in step S320 can include the following step S323.

Step S323: taking, based on the single-drug knowledge graph, a use-weight of each drug in the candidate drug set relative to the disease of the patient as the matching degree score of the each drug in the candidate drug set for the disease of the patient.

For example, in some embodiments, in the single-drug knowledge graph, the usage number of the drug entity U to treat the disease entity D (the usage number can be used to indicate the use-weight of the drug entity U relative to the disease entity D) is further recorded between the disease entity D (disease) and the drug entity U (drug) which have an indication relationship, so that the usage number of each drug in the candidate drug set relative to the disease of the patient can be taken as the matching degree score of the each drug in the candidate drug set for the disease of the patient, and further, the plurality of candidate drugs in the candidate drug set can be sorted from high to low according to the matching degree scores.

For example, in the case where the “determining the matching degree score of each drug in the candidate drug set for the disease of the patient based on the single-drug knowledge graph” in step S320 includes step S323, the drug recommendation method 10 can further include: increasing, based on a selection condition of the target recommended drug, a use-weight of a selected target recommended drug relative to the disease of the patient in the single-drug knowledge graph. Therefore, the single-drug knowledge graph can be updated and improved, which is beneficial to improving the accuracy and feasibility of drug recommendation. For example, in some embodiments, in the single-drug knowledge graph, the usage number of the drug entity U to treat the disease entity D (the usage number can be used to indicate the use-weight of the drug entity U relative to the disease entity D) is further recorded between the disease entity D (disease) and the drug entity U (drug) which have an indication relationship, so that the usage number of the selected target recommended drug relative to the disease of the patient can be increased by 1 in the single-drug knowledge graph according to the selection condition of the target recommended drugs, so as to increase the use-weight of the selected target recommended drug relative to the disease of the patient in the single-drug knowledge graph.

For example, in some embodiments, the drug combination knowledge graph can be used for recording drug combination habits of each disease (if any). For example, each candidate combined prescription in the candidate drug set is regarded as a “drug”, and based on the above steps S310 to S320, the related operation of step S300 can be realized (of course, the “single-drug knowledge graph” in step S320 is correspondingly replaced with the “drug combination knowledge graph”). For example, the drug recommendation method 10 can further include: increasing, based on a selection condition of the target recommended drug, a use-weight of a selected target recommended drug (i.e., a selected target recommended combined prescription) relative to the disease of the patient in the drug combination knowledge graph. Therefore, the drug combination knowledge graph can be updated and improved, which is beneficial to improving the accuracy and feasibility of drug recommendation.

Step S330: taking a drug conforming to a predetermined sorting rule in the candidate drug set as the target recommended drug.

For example, in some embodiments, the predetermined sorting rule may be sorting from high to low according to the matching degree scores (that is, in step S320, each drug in the candidate drug set is sorted from high to low according to the matching degree score). In this case, in step S330, the top N drugs in the candidate drug set can be taken as the target recommended drugs, where N is an integer greater than 0, and the specific value of N can be set according to actual needs. For example, in some other embodiments, the predetermined sorting rule may be sorting from low to high according to the matching degree scores (that is, in step S320, each drug in the candidate drug set is sorted from low to high according to the matching degree score). In this case, in step S330, the last N drugs in the candidate drug set can be taken as the target recommended drugs, where N is an integer greater than 0, and the specific value of N can be set according to actual needs. It should be noted that the predetermined sorting rule is not limited in the embodiments of the present disclosure.

For example, in some embodiments, in order to avoid generating a huge recommendation result while ensuring that the user has enough choices, the value range of N can be set to, for example, [3, 10] or [3, 5], etc., without being limited in the embodiments of the present disclosure.

Step S400: providing the target recommended drug.

For example, in some embodiments, step S400 can include: providing a plurality of medication schemes, wherein each medication scheme includes at least one drug. For example, each medication scheme can be a single drug or a drug combination. For example, the drug combination can be a combined prescription (including at least two drugs); the drug combination can also include a plurality of single drugs, and these single drugs are used to treat different diseases. For example, in some embodiments, each medication scheme further includes a prompt on the usage and dosage of each drug therein, which is used for reminding a user how to use the each drug.

For example, in some embodiments, in step S400, the target recommended drugs can be presented to the user in the form of text. For example, in some embodiments, the target recommended drugs can be selected by the user (e.g., a doctor, a patient, etc.), according to his/her own professional experience, medication habits, etc.

For example, in some embodiments, as shown in FIG. 6, before step S400, the drug recommendation method can further include step S390: performing, based on the drug knowledge graph and the patient information, a safety check on the target recommended drug, so as to obtain a check report of the target recommended drug.

For example, in some embodiments, the relevant operation in step S390 can be performed according to the drug knowledge graph (e.g., a single-drug knowledge graph). For example, in some embodiments, as shown in FIG. 7, step S390 can include the following steps S391 and S392.

Step S391: determining at least one selected from the group consisting of prohibition information, caution information and allergy information of the target recommended drug by inquiring the drug knowledge graph.

Step S392: matching the at least one selected from the group consisting of the prohibition information, the caution information and the allergy information of the target recommended drug with the patient information, so as to obtain the check report of the target recommended drug, wherein in the case where the at least one selected from the group consisting of the prohibition information, the caution information and the allergy information of the target recommended drug successfully matches with the patient information, the check report of the target recommended drug comprises at least a corresponding one selected from the group consisting of a prohibition reminder, a caution reminder and an allergy reminder.

For example, in some embodiments, a knowledge graph query and search algorithm can be used to perform the operation of step S391.

For example, in some embodiments, the patient information used in step S392 can include the patient features, such as the population attribute of the patient, the concomitant/potential disease or disorder information of the patient, the diagnosis and treatment condition of the patient, the medication condition of the patient (e.g., drug allergy history, etc.), etc. The embodiments of the present disclosure include but are not limited thereto.

Hereinafter, for convenience of description, the prohibition information of the target recommended drug, the caution information of the target recommended drug and the allergy information of the target recommended drug are collectively referred to as a “first type of information”, and the patient information used in step S392 is collectively referred to as a “second type of information”.

For example, in some embodiments, two types of information (i.e., the first type of information and the second type of information) can be matched one by one using a semantic similarity algorithm. For example, in some embodiments, firstly, word embedding can be performed on the first type of information and the second type of information, respectively, so as to correspondingly generate a first embedding vector A (“vector A” for short) and a second embedding vector B (“vector B” for short), and the vector A and the vector B are both numerical vectors. Word embedding can be viewed as a mapping relationship, which can map or embed a word in the text space into a numerical vector space by using a certain method. That is, word embedding can express words and a complete sentence in the form of vectors. Then, the vector A and the vector B can be inputted to at least one similarity model, and each similarity model outputs a similarity feature between the vector A and the vector B. The larger the value of the similarity feature, the more similar the word or sentence corresponding to the vector A and the word or sentence corresponding to the vector B. For example, commonly used similarity models include cosine similarity model, Jaccard similarity model, editing distance (Levenshtein) similarity model, word mover's distance (WMD) similarity model, and deep structured semantic model (DSSM), etc. It should be noted that the embodiment of the present disclosure does not limit the number of the similarity matching models being used. For example, in some examples, 5 similarity matching models can be used, and there may be 5 similarity features between the vector A and the vector B. For example, in some other examples, 3 similarity matching models can be used, and there may be 3 similarity features between the vector A and the vector B. It should be understood that in the case where a plurality of similarity models are adopted, the similarity features outputted by the plurality of similarity models can be weighted and summed (the respective weights can be set according to actual needs) as the final similarity feature; or, the plurality of similarity models can be used by means of setting hierarchical thresholds. For example, a similarity model M1 is adopted in the first hierarchy, and in the case where the similarity value calculated by using the similarity model M1 is greater than the threshold T1 set for the first hierarchy, it is directly considered as matching; otherwise, the second hierarchy is entered, and a similarity model M2 is adopted for calculation, and so on.

The similarity matching models mentioned above are briefly introduced below.

(1) Cosine similarity. Cosine similarity indicates the difference between two individuals by the cosine value of the angle between the vectors. The closer the cosine value is to 1, the more similar the two vectors A and B are. The following formula is usually used to calculate cosine similarity (also referred to as cosine distance).

$$similarity = cos ( θ ) = \frac{A \cdot B}{‖ A ‖ ‖ B ‖} = \frac{{\sum }_{i = 1}^{n} {A}_{i} \times {B}_{i}}{\sqrt{{\sum }_{i = 1}^{n} {A}_{i}^{2}} \times \sqrt{{\sum }_{i = 1}^{n} {B}_{i}^{2}}}$$

(2) Jaccard distance. Jaccard distance indicates the discrimination degree of between two sets by the proportion of different elements in all elements in the two sets. Jaccard distance can be expressed by, for example, the following formula, where J(A, B) is the Jaccard similarity coefficient.

$${d}_{j} ( A , B ) = 1 - J ( A , B ) = \frac{\vert A ⋃ B \vert - \vert A ⋂ B \vert}{\vert A ⋃ B \vert}$$

(3) Editing distance, also known as Levenshtein distance. Editing distance refers to the minimum number of operations required to convert string A into string B by using character operations. The permitted character operations include modifying a character, inserting a character, and deleting a character. Generally speaking, the smaller the editing distance between two strings, the more similar they are. If the two strings are the same, the editing distance therebetween is 0.

(4) Word mover's distance (WMD). The WMD refers to considering the similarity between two documents through the whole documents and measuring the semantic similarity of documents by finding the minimum cumulative distance that all words in one document need to travel to exactly match the other document.

(5) DSSM model. DSSM is a deep semantic matching model, which maps the matched two items into a low-dimensional space, and the correlation problem is transformed into the distance between low-dimensional space vectors. The model can not only predict the semantic similarity of two sentences, but also obtain the low-dimensional semantic vector expressions of the sentences.

It should be noted that the similarity matching models used in the embodiments of the present disclosure may not be limited to the similarity matching models described above, and other similarity matching models can also be used, as long as the same or similar technical effects can be achieved, namely, as long as the similarity between two vectors can be calculated. And the embodiments of the present disclosure do not specifically limit the similarity matching models. In addition, the embodiments of the present disclosure do not limit the number of the similarity matching models being used, which can be set according to actual needs.

For example, in some embodiments, in the case where the similarity feature between the vector A and the vector B satisfies a certain threshold condition, it is considered that the vector A and the vector B can be successfully matched. In this case, prohibition or caution reminders concerning, for example, disease, medical history, diagnosis and treatment condition, age attribute, physiological stage, job, etc., as well as allergy reminders, are provided for a relevant drug in the target recommended drugs, that is, a check report of a relevant drug in the target recommended drugs is generated. For example, in some examples, in the case where the prohibition or caution information of a certain drug in the target recommended drugs includes “prohibition and caution for pregnant woman”, the vector A can correspond to “pregnant woman”; and if the patient features include the label “pregnant woman” at the same time (that is, the vector B can also correspond to “pregnant woman”), then the vector A and the vector B can be successfully matched; therefore, this drug in the target recommended drugs can be reminded of prohibition and caution (for example, the words “prohibition and caution” is generated).

For example, in some embodiments, as shown in FIG. 7, step S390 can further include step S393: in the case where the target recommended drug includes a drug combination, determining incompatibility information of various drugs in the drug combination by inquiring the drug knowledge graph, determining whether an incompatibility is existed between the various drug in the drug combination based on the incompatibility information of the various drugs in the drug combination, and providing an incompatibility reminder in response to that an incompatibility is existed between the various drugs in the drug combination, wherein the check report of the target recommended drug further includes the incompatibility reminder of the drug combination.

For example, in some embodiments, the case in which the target recommended drug includes a drug combination generally refers to that: the drug combination is a combined prescription (including at least two drugs); or, the drug combination includes a plurality of single drugs, and these single drugs are used for treating different diseases of the same patient. For example, in some embodiments, in step S393, a semantic similarity algorithm can also be used for matching the incompatibility information with the named entity of each drug in the drug combination one by one, so as to judge whether an incompatibility is existed between various drugs in the drug combination. For example, the semantic similarity algorithm described above can be used for matching the incompatibility information of any drug in the drug combination with the named entities of other drugs in the drug combination one by one. If the matching is successful, it indicates that an incompatibility is existed between the any drug and the corresponding drug successfully matched with the any drug; otherwise, there is no incompatibility between the drugs in the drug combination. It should be understood that the knowledge in the drug combination knowledge graph is usually correct and reliable, and there is usually no incompatibility between the various drugs in the combined prescription. Therefore, in the case where the drug combination is a combined prescription, step S393 can be omitted.

For example, in some embodiments, step S390 can further include: in the case where the target recommended drug includes a drug combination, performing the safety check on the drug combination by using medical big data, such as an electronic medical record, etc. For example, in some embodiments, the drug combined prescriptions (actually, that is, combined prescriptions, which are called “drug combined prescriptions” here to distinguish them from the combined prescription actually provided, that is, the recommended prescriptions) appeared in the data can be traversed, various drugs appeared in the drug combined prescriptions are taken as nodes, initial edge-weights between various drug are set to 0, and the edge-weight between two drugs every time a combination of the two drugs appears in the data, so as to form a complicated network with weights. Then, based on the complicated network with weights, the safety of the drug combination can be determined by a graph search algorithm, and the check report of the target recommended drug further includes a safety determination result of the drug combination. For example, if a sub-graph corresponding to the recommended prescription can be inquired and the larger the edge-weights between the drugs in the recommended prescription are (for example, greater than a certain threshold T), it indicates that the more safe and more reasonable the recommended prescription is. If a sub-graph corresponding to the recommended prescription cannot be inquired and the smaller the edge-weights between the drugs in the recommended prescription are (for example, not greater than the above threshold T), it indicates that the less safe and less reasonable the recommended prescription is, and a corresponding safety and reasonability reminder may be provided. By using this scheme to perform the safety check on drug combination (especially combined prescription) in the target recommended drugs, the drug knowledge graph is not needed to rely on, and meanwhile, the information contained is authentic, authoritative, and real-time, and the evaluation index can be quantified.

For example, in the embodiments of the present disclosure, the adverse medication information of a drug, such as prohibition information, caution information, allergy information and incompatibility information, etc., is highly structured, so as to form a knowledge graph and perform the safety check on the target recommended drug in combination with a related algorithm in the field of natural language processing, so that the safety check on the target recommended drug can be more efficient and convenient. By performing the safety check, the safety of the target recommended drug can be effectively improved, thus facilitating a user (e.g., a doctor) to issue a more safe prescription, and further ensuring the medication safety of the patients.

For example, in some embodiments, as shown in FIG. 6, based on step S390, step S400 can further include: providing the check report of the target recommended drug, while providing the target recommended drug. That is to say, the target recommended drug and the check report of the target recommended drug are provided at the same time.

For example, in some embodiments, the user can obtain the target recommended drug in step S300 and the check report in step S390. For example, in some examples, the target recommended drug and the check report can be presented to the user in the form of texts (e.g., a medication report), and some graphics, charts, thumbnails, etc., can further be added on the basis of the texts, thus facilitating the user to obtain information more intuitive.

For example, in some embodiments, step S400 can further include providing the judgment result of the drug combination necessity. And therefore, the user can further obtain the judgment result of the drug combination necessity in step S150. For example, in some embodiments, step S400 can further include providing a recommendation reason for the target recommended drug.

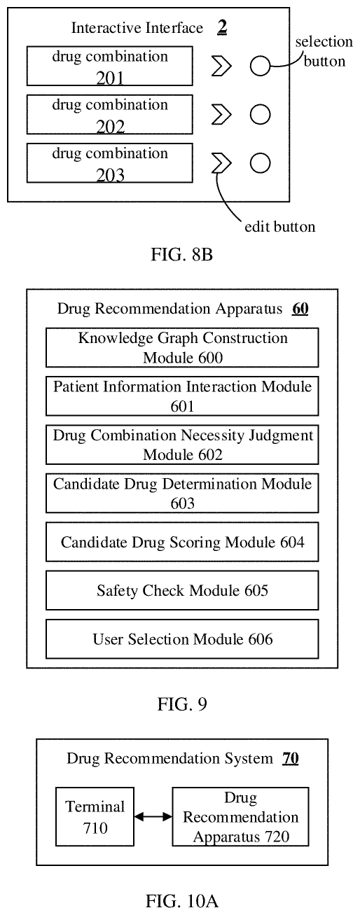

For example, in some embodiments, the target recommended drugs can be selected by the user according to the check report. For example, in some examples, the user can select an optimal drug from the plurality of drugs in the target recommended drugs as an official prescription, based on his/her own professional experience and referring to the check report described above. For example, in some other examples, the user can make an adjustment to the drug combination in the target recommended drugs (e g., deleting one or more drugs in the drug combination, or adding one or more drugs to the drug combination) and take the adjusted drug combination as an official prescription, based on his/her own professional experience and referring to the check report described above. For example, in some other embodiments, the user can make a selection from the target recommended drugs based on the judgment result and the check report. For example, in some examples, the user can select an optimal drug from the plurality of drugs in the target recommended drug as an official prescription, based on his/her own professional experience and comprehensively considering the judgment result and the check report. For example, in some other examples, the user can make an adjustment to the drug combination in the target recommended drugs (e g., deleting one or more drugs in the drug combination, or adding one or more drugs to the drug combination) and take the adjusted drug combination as an official prescription, based on his/her own professional experience and comprehensively considering the judgment result and the check report. FIG. 8A is a schematic diagram of an interactive interface provided by at least some embodiments of the present disclosure. For example, in the interactive interface 1 shown in FIG. 8A, three single drugs (namely, drug 101, drug 102, and drug 103) are provided, and the user can click on each single drug to obtain the medication report of the single drug (including usage and dosage, and a check report, etc.). For example, the user can also click any selection button to take the single drug corresponding to the selection button as an official prescription.

FIG. 8B is a schematic diagram of another interactive interface provided by at least some embodiments of the present disclosure. For example, in the interactive interface 2 shown in FIG. 8B, three drug combinations (i.e., drug combination 201, drug combination 202, and drug combination 203) are provided, and the user can click on each drug combination to obtain the medication report of the drug combination (including usage and dosage of various drugs in the drug combination, and a check report of the drug combination, etc.). For example, the user can also click an edit button to enter the edit mode of a corresponding drug combination. In the edit mode, the user is allowed to delete one or more drugs in the drug combination, or add one or more drugs into the drug combination. For example, the user can also click any selection button to take the drug combination corresponding to the selection button as an official prescription.

For example, in some embodiments, as shown in FIG. 6, the drug recommendation method 10 can further include step S500: updating the drug knowledge graph based on a selection condition of the target recommended drug.

For example, in some embodiments, step S500 can include: increasing, based on a selection condition of the target recommended drug, a use-weight of a selected target recommended drug relative to the disease of the patient in the single-drug knowledge graph. Therefore, the single-drug knowledge graph can be updated and improved, which is beneficial to improving the accuracy and feasibility of drug recommendation.

For example, in some embodiments, step S500 can further include: in the case where a drug combination in the target recommended drugs is adjusted and then selected, updating at least one of the single-drug knowledge graph and the drug combination knowledge graph based on the patient information and the adjusted drug combination (i.e., the official prescription). For example, in some embodiments, the current patient information and official prescription can be taken as an electronic medical record, and at least one of the current single-drug knowledge graph and drug combination knowledge graph can be updated based on this electronic medical record, and for example, the updating method can be referred to the constructing method of the related knowledge graph, which is not repeated here. For example, in some embodiments, the binary classification model used in step S150 can also be updated based on the electronic medical record; and for example, the updating method can be referred to the constructing method of the binary classification model, which is not repeated here. Therefore, in the process of performing the drug recommendation method 10, the knowledge base (e.g., the single-drug knowledge graph, the drug combination knowledge graph, and the binary classification model, etc.) on which the drug recommendation method 10 relies can be revised, updated and improved, which is beneficial to improving the accuracy and feasibility of drug recommendation.

It should be noted that, in the embodiments of the present disclosure, the above-mentioned steps (e.g., step S000, step S100, step S150, step S200, step S300, step S390, step S400 and step S500, etc.) can be performed sequentially, or be performed in other adjusted sequence, and some or all of the operations in the above steps can also be performed in parallel. The embodiments of the present disclosure do not limit the execution sequence of the steps, which can be adjusted according to actual situations. For example, in the embodiments of the present disclosure, the above steps can be executed on a separate server (e.g., cloud server, etc.), or can also be executed on a local terminal; alternatively, one part of the above steps can be executed on a local terminal, and the other part of the above steps can be executed on a remote server. The embodiments of the present disclosure are not limited to these cases. For example, in some embodiments, some of the above steps in the drug recommendation method 10 described above can be selectively performed, or some additional steps other than to the above steps can be performed, which are not specifically limited in the embodiments of the present disclosure.

At least some embodiments of the present disclosure further provide a drug recommendation apparatus. FIG. 9 is a schematic block diagram of a drug recommendation apparatus provided by at least one embodiment of the present disclosure.

For example, in some embodiments, as shown in FIG. 9, the drug recommendation apparatus 60 can include a knowledge graph construction module 600, a patient information interaction module 601, a drug combination necessity judgment module 602, a candidate drug determination module 603, a candidate drug scoring module 604, a safety check module 605, and a user selection module 606.

For example, in some embodiments, the knowledge graph construction module 600 is configured to construct a drug knowledge graph. In other words, the knowledge graph construction module 600 can be configured to perform step S000 in the aforementioned drug recommendation method 10. For example, the specific operation procedure and details of the knowledge graph construction module 600 can be referred to the related description of the aforementioned step S000, which is not repeated here. For example, in some embodiments, the knowledge graph construction module 600 is further configured to update the drug knowledge graph based on a selection condition of the target recommendation drug. In other words, the knowledge graph construction module 600 can be further configured to perform step S500 in the aforementioned drug recommendation method 10. For example, the specific operation procedure and details of the knowledge graph construction module 600 can be referred to the related description of the aforementioned step S500, which is not repeated here.

For example, in some embodiments, the patient information interaction module 601 is configured to obtain patient information. In other words, the patient information interaction module 601 can be configured to perform step S100 in the aforementioned drug recommendation method 10. For example, the specific operation procedure and details of the patient information interaction module 601 can be referred to the related description of the aforementioned step S100, which is not repeated here.

For example, in some embodiments, the drug combination necessity judgment module 602 is configured to judge a drug combination necessity based on the patient information, so as to obtain a judgment result of the drug combination necessity, wherein the judgment result of the drug combination necessity includes needing drug combination or not needing drug combination. In other words, the drug combination necessity judgment module 602 can be configured to perform step S150 in the aforementioned drug recommendation method 10. For example, the specific operation procedure and details of the drug combination necessity judgment module 602 can be referred to the related description of the aforementioned step S150, which is not repeated here.

For example, in some embodiments, the candidate drug determination module 603 is configured to determine a candidate drug set based on a drug knowledge graph and the patient information. In other words, the candidate drug determination module 603 can be configured to perform step S200 in the aforementioned drug recommendation method 10. For example, in some embodiments, the drug knowledge graph includes a single-drug knowledge graph and a drug combination knowledge graph, and the candidate drug determination module 603 is configured to determine, in response to that the judgment result of the drug combination necessity is not needing drug combination, the candidate drug set based on the single-drug knowledge graph and the patient information (that is, performing the related operation of step S210), or to determine, in response to that the judgment result of the drug combination necessity is needing drug combination, the candidate drug set based on the drug combination knowledge graph and the patient information (that is, performing the related operation of step S220). For example, the specific operation procedure and details of the candidate drug determination module 603 can be referred to the related description of the aforementioned step S150, which is not repeated here.

For example, in some embodiments, the candidate drug scoring module 604 is configured to score each drug in the candidate drug set and determine a target recommended drug based on a scoring result. In other words, the candidate drug scoring module 604 can be configured to perform step S300 in the aforementioned drug recommendation method 10. For example, the specific operation procedure and details of the candidate drug scoring module 604 can be referred to the related description of the aforementioned step S300, which is not repeated here.

For example, in some embodiments, the safety check module 605 is configured to perform a safety check on the target recommended drug based on the drug knowledge graph and the patient information, so as to obtain a check report of the target recommended drug. In other words, the safety check module 605 can be configured to perform step S390 in the aforementioned drug recommendation method 10. For example, the specific operation procedure and details of the safety check module 605 can be referred to the related description of the aforementioned step S390, which is not repeated here.

For example, in some embodiments, the user selection module 606 is configured to provide the target recommended drug. In other words, the user selection module 606 can be configured to perform step S400 in the aforementioned drug recommendation method 10. For example, the specific operation procedure and details of the user selection module 606 can be referred to the related description of the aforementioned step S400, which is not repeated here.

It should be noted that the knowledge graph construction module 600, the patient information interaction module 601, the drug combination necessity judgment module 602, the candidate drug determination module 603, and the candidate drug scoring module 604, the safety check module 605, the user selection module 606, etc., in the drug recommendation apparatus 60 can be implemented by software, hardware, firmware, or any combination thereof. For example, the knowledge graph construction module 600, the patient information interaction module 601, the drug combination necessity judgment module 602, the candidate drug determination module 603, the candidate drug scoring module 604, the safety check module 605, and the user selection module 606 can be implemented as a knowledge graph construction circuit, a patient information interaction circuit, a drug combination necessity judgment circuit, a candidate drug determination circuit, a candidate drug scoring circuit, a safety check circuit and a user selection circuit, respectively. It should be noted that the embodiments of the present disclosure do not limit the specific implementation manners thereof. It should also be noted that, corresponding to the drug recommendation method 10 provided by the embodiments of the present disclosure, some of the above modules in the drug recommendation apparatus can be omitted according to actual needs. For example, at least one of the knowledge graph construction module 600, the drug combination necessity judgment module 602 and the safety check module 605 may be omitted.

It should be understood that the drug recommendation apparatus 60 provided by the embodiments of the present disclosure can be used to implement the aforementioned drug recommendation method 10, and thus, can also achieve the same technical effects as the aforementioned drug recommendation method 10, which is not repeated here.

It should be noted that, in the embodiments of the present disclosure, the drug recommendation apparatus 60 can include more or less software, hardware and firmware, and the connection relationships between software, hardware and firmware are not limited, which can be determined according to actual needs. The specific formation manner of software, hardware and firmware is not limited, which can be formed of digital chips, be formed in a manner of a combination of a processor and a memory, or be formed in any other suitable manner.

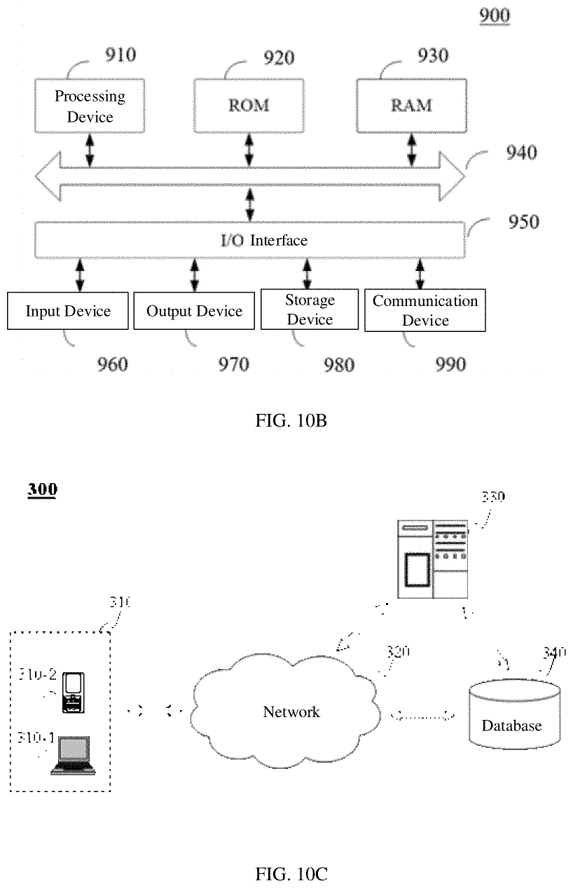

At least some embodiments of the present disclosure further provide a drug recommendation system. FIG. 10A is a schematic block diagram of a drug recommendation system provided by at least some embodiments of the present disclosure. For example, as shown in FIG. 10A, the drug recommendation system 70 includes a terminal 710 and a drug recommendation apparatus 720, and the terminal 710 and the drug recommendation apparatus 720 are in signal connection with each other.

For example, the above method can be executed on the server, and the result is sent to the terminal; or, the method can also be executed on the terminal.

For example, in some embodiments, the terminal 710 is configured to send request data to the drug recommendation apparatus 720. For example, in some embodiments, the request data can include patient information data or a path address of the patient information data. For example, in some embodiments, the patient information data includes one or more of the following terms: a physical examination report, an electronic medical record, and a question-and-answer record of a patient. For example, in some examples, the physical examination report can be sent to the drug recommendation apparatus 720 via various physical examination devices in the physical examination system; and in this case, the terminal 710 includes various physical examination devices in the physical examination system. For example, in some other examples, the physical examination report can be sent to the drug recommendation apparatus 720 by the used via the terminal 710. For example, the physical examination report of an electronic version can be directly sent to the drug recommendation apparatus 720, and the physical examination report of an the paper version can be converted to an electronic version and then sent to the drug recommendation apparatus 720.

For example, in some embodiments, the drug recommendation apparatus 720 is configured to: obtain patient information based on the request data; determine a candidate drug set based on a drug knowledge graph and the patient information; score each drug in the candidate drug set and determine a target recommended drug based on a scoring result; and provide the target recommended drug to the terminal. That is to say, the drug recommendation apparatus 720 can be used to execute the drug recommendation method 10 provided by any embodiment of the present disclosure, which is not repeated here. For example, the drug recommendation apparatus 720 being configured to obtain the patient information based on the request data can include that the drug recommendation apparatus 720 is configured to obtain the patient information according to the patient information data in the request data or according to the path address of the patient information data in the request data.

For example, in some examples, the terminal 710 included in the drug recommendation system 70 can be implemented as a client terminal (e.g., a mobile phone, a computer, etc.), and the drug recommendation apparatus 720 can be implemented as a server terminal (e.g., a server, etc.).

For example, in some embodiments, in addition to the terminal 710 and the drug recommendation apparatus 720, the drug recommendation system 70 can further include a knowledge base server (not shown in FIG. 10A) storing a drug knowledge graph (e.g., including a single-drug knowledge graph and/or a drug combination knowledge graph). The knowledge base server is in signal connection with the drug recommendation apparatus 720, and is configured, in response to request information of the drug recommendation apparatus 720, to return data corresponding to the request information in the drug knowledge graph to the drug recommendation apparatus 720. It should be noted that, in the case where the drug recommendation system 70 does not include the knowledge base server 730, the data in the drug knowledge graph can be directly stored on the drug recommendation apparatus 720 or stored on any other storage device additionally provided. Or, the drug recommendation apparatus 720 itself establishes the drug knowledge graph and then the drug knowledge graph is stored on the drug recommendation apparatus 720 or stored on any other storage device additionally provided. The embodiments of the present disclosure are not limited to these cases.

For example, in some embodiments, the drug recommendation system 70 may further include a physical examination system (not shown in FIG. 10A), which is configured to provide patient information to the drug recommendation apparatus 720. For example, the physical examination system may include various medical examination devices and this medical examination devices may generate a medical examination report (including patient information) and provide it to the drug recommendation apparatus 720.

FIG. 10B is a schematic block diagram of a terminal provided by at least some embodiments of the present disclosure. For example, in some embodiments, the terminal is a display terminal 900, which can be applied in the drug recommendation system provided by the embodiments of the present disclosure. For example, the display terminal 900 can send request data to the drug recommendation apparatus and display the target recommended drug provided by the drug recommendation apparatus to the user. It should be noted that the terminal shown in FIG. 9 is merely an example of the display terminal 900, which will not bring any limitation to the function and scope of use of the embodiments of the present disclosure.

As shown in FIG. 10B, the display terminal 900 can include a processing device (e.g., a central processing unit, a graphics processing unit, etc.) 910, which can perform various appropriate actions and processes according to the program stored in the read-only memory (ROM) 920 or the program loaded from a storage device 980 to the random access memory (RAM) 930. On the RAM 930, various programs and data required for the operations of the display terminal 900 are also stored. The processing device 910, the ROM 920, and the RAM 930 are connected with each other through a bus 940. The input/output (I/O) interface 950 is also connected to the bus 940.

Generally, the following devices can be connected to the I/O interface 950: an input device 960, including, for example, a touch screen, a touch pad, a keyboard, a mouse, a camera, a microphone, an accelerometer, a gyroscope, etc.; an output device 970, including, for example, a liquid crystal display (LCD), a speaker, a vibrator, etc.; a storage device 980, including, for example, a magnetic tape, a hard disk, etc.; and a communication device 990. The communication device 990 can allow the display terminal 900 to perform a wireless or wired communication with other electronic devices, so as to exchange data. Although FIG. 10B shows the display terminal 900 having various devices, it should be understood that it is not required to implement or have all the illustrated devices, and the display terminal 900 can alternatively implement or have more or fewer devices.

It should be understood that, in some embodiments, the above terminal can also be used to implement the aforementioned drug recommendation method 10.

FIG. 10C is a schematic block diagram of another drug recommendation system provided by at least some embodiments of the present disclosure. For example, as shown in FIG. 10C, the drug recommendation system can include a user terminal 310, a network 320, a drug recommendation apparatus 330, and a database 340.

For example, the user terminal 310 can be a computer 310-1 or a portable terminal 310-2 as shown in FIG. 10C. It can be understood that the user terminal can also be any other type of electronic device, which is capable of receiving, processing, and displaying data. The user terminal can include, but is not limited to, a desktop computer, a notebook computer, a tablet computer, a smart home device, a wearable device, in-vehicle electronic device, medical electronic device, etc.

For example, the network 320 can be a single network, or a combination of at least two different networks. For example, the network 320 can include, but is not limited to, one or any combination of a local area network, a wide area network, a public network, a private network, the Internet, a mobile communication network, etc.

For example, the drug recommendation apparatus 330 can be a single server or a server group, and the servers in the server group are connected through a wired network or a wireless network. The wired network, for example, can communicate by means of twisted pair, coaxial cable or optical fiber transmission, etc. The wireless network, for example, can adopt a communication mode such as 3G/4G/5G mobile communication network, Bluetooth, Zigbee or WiFi, etc. The present disclosure does not limit the type and function of the network. The server group can be centralized, such as a data center, or can be distributed. The server can be local or remote. For example, the drug recommendation apparatus 330 can be a general-purpose server or a dedicated server, and can be a virtual server or a cloud server, etc.

For example, database 340 can be used for storing various data which is used, generated, and outputted from the operations of the user terminal 310 and the drug recommendation apparatus 330. The database 340 can be connected or communicated with the drug recommendation apparatus 330 or with a part of the drug recommendation apparatus 330 via the network 320, or can be directly connected or communicated with the drug recommendation apparatus 330, or can be connected or communicated with the drug recommendation apparatus 330 via a combination of the above two manners. In some embodiments, the database 340 can be an independent device. In some other embodiments, the database 340 can also be integrated in at least one of the user terminal 310 and the drug recommendation apparatus 330. For example, the database 340 can be set in the user terminal 310, or can be set in the drug recommendation apparatus 330. For another example, the database 340 can also be distributed, one part of the database 340 is set in the user terminal 310, and the other part of the database 340 is set in the drug recommendation apparatus 330.

For example, in some examples, firstly, the user terminal 310 (e.g., the mobile phone of the user) can send request data to the drug recommendation apparatus 330 via the network 320 or other technology (e.g., Bluetooth communication, infrared communication, etc.). Next, the drug recommendation apparatus 330 obtains the patient information based on the request data. For example, the request data includes patient information data or a path address of the patient information data. Then, the drug recommendation apparatus 330 determines a candidate drug set based on a drug knowledge graph and the patient information. Next, the drug recommendation apparatus 330 scores each drug in the candidate drug set, determines the target recommended drug according to the scoring result, and then sends the target recommended drug to the user terminal 310. Finally, the user terminal 310 displays the target recommended drug after receiving the target recommended drug from the drug recommendation apparatus 330.

For example, for a detailed description of the specific implementation process and details of the drug recommendation method, reference can be made to the related description of the embodiments of the drug recommendation method 10, which will not be repeated here.

The drug recommendation system provided by the embodiments of the present disclosure can implement the drug recommendation method 10 provided by the foregoing embodiments, and can also achieve similar technical effects as the drug recommendation method 10 provided by the foregoing embodiment, which will not be repeated here.

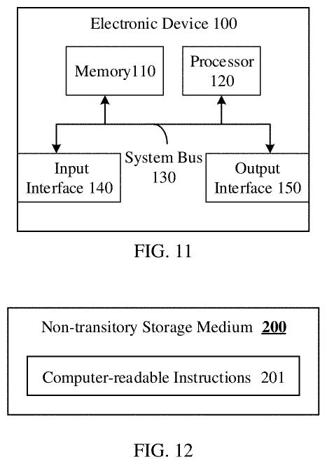

At least some embodiments of the present disclosure further provide an electronic device. FIG. 11 is a schematic block diagram of an electronic device provided by at least some embodiments of the present disclosure. For example, as illustrated in FIG. 11, the electronic device 100 includes a memory 110 and a processor 120.

For example, the memory 110 is configured to non-transitorily store computer readable instructions, and the processor 120 is configured to execute the computer readable instructions. For example, upon the computer readable instructions being executed by the processor 220, the drug recommendation method provided by any one of the embodiments of the present disclosure is executed.

For example, the memory 110 and the processor 120 may communicate with each other directly or indirectly. For example, in some examples, as illustrated in FIG. 11, the electronic device 100 can further include a system bus 130, and the memory 110 and the processor 120 can communicate with each other through the system bus 130. For example, the processor 120 can access the memory 110 through the system bus 130. For example, in some other examples, components, such as the memory 110 and the processor 120, etc., can communicate with each other via network connection. The network can include a wireless network, a wired network, and/or any combination of the wireless network and the wired network. The network can include a local area network, the Internet, a telecommunication network, Internet of Things based on the Internet and/or the telecommunication network, and/or any combination of the above networks, etc. The wired network, for example, can communicate by means of twisted pair, coaxial cable or optical fiber transmission, etc. The wireless network, for example, can adopt a communication mode such as 3G/4G/5G mobile communication network, Bluetooth, Zigbee or WiFi, etc. The present disclosure does not limit the type and function of the network.

For example, the processor 120 can control other components in the electronic device to realize desired functions. The processor 120 can be an element having data processing capability and/or program execution capability, such as a central processing unit (CPU), a tensor processing unit (TPU), or a graphics processing unit (GPU). The CPU can have an X86 or ARM architecture, etc. The GPU can be integrated directly on the motherboard alone or built into the Northbridge chip of the motherboard. The GPU can also be built into the CPU.

For example, the memory 110 can include one or a plurality of computer program products, and the computer programs can include a computer readable storage medium of diverse forms, such as a volatile memory and/or a non-volatile memory. The volatile memory, for instance, can include a random access memory (RAM) and/or a cache, etc. The non-volatile memory, for example, can include a read-only memory (ROM), a hard disk, an erasable programmable read-only memory (EPROM), a portable compact disk read-only memory (CD-ROM), a USB memory, or a flash memory, etc.

For example, one or a plurality of computer instructions can be stored on the memory 110, and the processor 120 can execute the computer instructions to realize various functions. The computer readable storage medium can also store various application programs and various data, such as the single-drug knowledge graph, the drug combination knowledge graph, the binary classification model, the recommended prescription, the official prescription, and various data used and/or generated by the application programs.

For example, upon some computer instructions stored in the memory 110 being executed by the processor 120, one or more steps in the drug recommendation method described above can be executed.

For example, as illustrated in FIG. 11, the electronic device 100 can further include an input interface 140 that allows an external device to communicate with the electronic device 100. For example, the input interface 140 can be configured to receive instructions from an external computer device or a user, etc. The electronic device 100 can further include an output interface 150 that allows the electronic device 100 to be connected with one or more external devices. For example, the electronic device 100 can output the recommended prescription, the check report and the like through the output interface 150. The external devices that communicate with the electronic device 100 through the input interface 240 and/or the output interface 250 can be included in an environment that provides a user interface of any type with which the user can interact with the external devices. Examples of the types of user interfaces include graphical user interface (GUI), natural user interface, etc. For instance, the GUI can receive an input from a user via an input device such as a keyboard, a mouse, a remote controller, and the like, and provide an output on an output device such as a display. In addition, the natural user interface can enable a user to interact with the accent detection device 200 in a manner that is not constrained by input devices such as keyboards, mice and remote controllers. In contrast, the natural user interface can rely on voice recognition, touch and stylus recognition, gesture recognition on and near the screen, aerial gesture, head and eye tracking, speech and semantics, vision, touch, gesture, and machine intelligence, etc.

Moreover, although the electrode device 100 is illustrated as an individual system in FIG. 11, it should be understood that the electrode device 100 can also be a distributed system and can also be deployed as a cloud facility (including public cloud or private cloud). Thus, for example, a plurality of devices can communicate with each other via network connection and execute the tasks that are described to be executed by the electrode device 100 together.

For example, for a detailed description of the implementation process of the drug recommendation method, reference may be made to the relevant description of the above-mentioned embodiments of the drug recommendation method 10, and the repeated descriptions are omitted here.

For example, in some examples, the electronic device can include, but is not limited to, a smart phone, a laptop, a tablet computer, and a desktop computer, etc.

It should be noted that the electrode device provided by the embodiments of the present disclosure is illustrative but not limitative, and the electrode device can also include other conventional components or structures according to actual application requirements. For instance, in order to implement necessary functions of the electrode device, those skilled in the art can set other conventional components or structures according to specific application scenarios, which are not limited in the embodiments of the present disclosure.

Technical effects of the electrode device provided by the embodiments of the present disclosure can be referred to the related description of the drug recommendation method in the above embodiments, and no further description will be given here.

At least some embodiments of the present disclosure further provide a non-transitory storage medium. FIG. 12 is a schematic diagram of a non-transitory storage medium provided by at least some embodiments of the present disclosure. For example, as illustrated in FIG. 12, the non-transitory storage medium 200 stores computer-readable instructions 201 non-transitorily, and upon the non-transitory computer-readable instructions 201 being executed by a computer (including a processor), the instructions for the drug recommendation method provided by any one of the embodiments of the present disclosure can be executed.

For example, one or more computer instructions may be stored on the non-transitory storage medium 200. Some computer instructions stored on the non-transitory storage medium 200 can be, for example, instructions for implementing one or more steps of the drug recommendation method described above.

For example, the non-transitory storage medium can include a storage component of a tablet, a hard disk of a personal computer, a random access memory (RAM), a read-only memory (ROM), an erasable programmable read-only memory (EPROM), a portable compact disk read-only memory (CD-ROM), a flash memory, or any combination of the above-mentioned storage media, or other suitable storage medium. For example, the non-transitory storage medium can also be the memory 110 shown in FIG. 11, and the relevant description can be referred to the foregoing content, which is not repeated here. For example, the non-transitory storage medium can be applied to the drug recommendation apparatus 720, and those skilled in the art can make a selection according to specific scenarios, which is not limited here.

Technical effects of the non-transitory storage medium provided by the embodiments of the present disclosure can be referred to the related description of the drug recommendation methods provided by the above embodiments, and no further description will be given here.

For the present disclosure, the following statements should be noted:

(1) The accompanying drawings related to the embodiment(s) of the present disclosure involve only the structure(s) in connection with the embodiment(s) of the present disclosure, and other structure(s) can be referred to common design(s).

(2) In case of no conflict, features in one embodiment or in different embodiments can be combined.

What have been described above are only specific implementations of the present disclosure, and the protection scope of the present disclosure is not limited thereto. Any changes or substitutions easily occur to those skilled in the art within the technical scope of the present disclosure should be covered in the protection scope of the present disclosure. Therefore, the protection scope of the present disclosure should be determined based on the protection scope of the claims.

What is claimed is:

1. A drug recommendation method, comprising:
obtaining patient information;
determining, based on a drug knowledge graph and the patient information, a candidate drug set;
scoring each drug in the candidate drug set and determining a target recommended drug based on a scoring result; and
providing the target recommended drug.

2. The drug recommendation method according to claim 1, wherein the drug knowledge graph comprises a single-drug knowledge graph, and
the determining, based on the drug knowledge graph and the patient information, the candidate drug set, comprises:
determining, based on the single-drug knowledge graph and the patient information, the candidate drug set.

3. The drug recommendation method according to claim 2, wherein the scoring each drug in the candidate drug set and determining the target recommended drug based on the scoring result, comprises:
determining a disease of a patient based on the patient information;
determining a matching degree score of each drug in the candidate drug set for the disease of the patient based on the single-drug knowledge graph, and sorting each drug in the candidate drug set base on the matching degree score; and
taking a drug conforming to a predetermined sorting rule in the candidate drug set as the target recommended drug.

4. The drug recommendation method according to claim 3, wherein the single-drug knowledge graph comprises a drug-indication-disease triple-tuple data set, and
the determining the matching degree score of each drug in the candidate drug set for the disease of the patient based on the single-drug knowledge graph, comprises:
representing all of the drug-indication-disease triple-tuple data set in the single-drug knowledge graph as a bipartite graph; and
performing a random walk in the bipartite graph based on a random walk algorithm, so as to calculate the matching degree score of each drug in the candidate drug set for the disease of the patient.

5. The drug recommendation method according to claim 4, wherein the bipartite graph comprises a plurality of drug nodes corresponding to all drugs in the single-drug knowledge graph, a plurality of disease nodes corresponding to all diseases in the drug knowledge graph, and a path connecting any drug node and any disease node which have an indication relationship, and
the performing the random walk in the bipartite graph based on the random walk algorithm, so as to calculate the matching degree score of each drug in the candidate drug set for the disease of the patient, comprises:
setting a random walk probability, and setting initial access probabilities of all nodes in the bipartite graph;
in each walk process, taking a disease node corresponding to the disease of the patient as a starting point to start walking, and upon walking to any node, determining whether to continue to walk or stop the present walk process based on the random walk probability, and in case of stopping the present walk process, calculating access probabilities of all nodes in the bipartite graph based on an iterative formula as follows:
$$PR ( i ) = \{ \begin{matrix}α * {\sum }_{j ∈ i n ( i )} \frac{PR ( j )}{\vert out ( j ) \vert} & if ( i \neq D ) \\ ( 1 - α ) + α * {\sum }_{j ∈ i n ( i )} \frac{PR ( j )}{\vert out ( j ) \vert} & if ( i = D )\end{matrix} ,$$
where PR (i) represents an access probability of a node i, α represents the random walk probability, in(i) represents a set of all nodes pointing to the node i, a node j is any node in the in(i), and out(j) represents a set of all nodes pointed to the node j; and
judging whether the above random walk process meets an iterative termination condition,
if the iterative termination condition is not met, repeating the above random walk process, and
if the iterative termination condition is met, stopping the above random walk process, and taking an access probability of a drug node corresponding to each drug in the candidate drug set as the matching degree score of the each drug in the candidate drug set for the disease of the patient.

6. The drug recommendation method according to claim 3, wherein the single-drug knowledge graph comprises a use-weight of a drug corresponding to each disease, and
the determining the matching degree score of each drug in the candidate drug set for the disease of the patient based on the single-drug knowledge graph, comprises:
taking, based on the single-drug knowledge graph, a use-weight of each drug in the candidate drug set relative to the disease of the patient as the matching degree score of the each drug in the candidate drug set for the disease of the patient.

7. The drug recommendation method according to claim 6, further comprising:
increasing, based on a selection condition of the target recommended drug, a use-weight of a selected target recommended drug relative to the disease of the patient in the single-drug knowledge graph.

8. The drug recommendation method according to claim 1, wherein the drug knowledge graph comprises a drug combination knowledge graph, and
the determining, based on the drug knowledge graph and the patient information, the candidate drug set, comprises:
determining, based on the drug combination knowledge graph and the patient information, the candidate drug set.

9. The drug recommendation method according to claim 8, wherein the patient information comprises a disease of a patient, and
the determining, based on the drug combination knowledge graph and the patient information, the candidate drug set, comprises:
inquiring all combined prescriptions having an indication relationship with the disease of the patient in the drug combination knowledge graph, so as to obtain the candidate drug set.

10. The drug recommendation method according to claim 1, further comprising:
judging a drug combination necessity based on the patient information, and obtaining a judgment result of the drug combination necessity, wherein the judgment result of the drug combination necessity comprises needing drug combination or not needing drug combination;
wherein the drug knowledge graph comprises a single-drug knowledge graph and a drug combination knowledge graph;
the determining, based on the drug knowledge graph and the patient information, the candidate drug set, comprises:
in response to that the judgment result of the drug combination necessity is not needing drug combination, determining, based on the single-drug knowledge graph and the patient information, the candidate drug set, or,
in response to that the judgment result of the drug combination necessity is needing drug combination, determining, based on the drug combination knowledge graph and the patient information, the candidate drug set.

11. The drug recommendation method according to claim 1, further comprising:
performing, based on the drug knowledge graph and the patient information, a safety check on the target recommended drug, so as to obtain a check report of the target recommended drug.

12. The drug recommendation method according to claim 11, wherein the performing, based on the drug knowledge graph and the patient information, the safety check on the target recommended drug, so as to obtain the check report of the target recommended drug, comprises at least one of the following operations:
determining at least one selected from the group consisting of prohibition information, caution information and allergy information of the target recommended drug by inquiring the drug knowledge graph; and matching the at least one selected from the group consisting of the prohibition information, the caution information and the allergy information of the target recommended drug with the patient information, so as to obtain the check report of the target recommended drug, wherein in a case where the at least one selected from the group consisting of the prohibition information, the caution information and the allergy information of the target recommended drug successfully matches with the patient information, the check report of the target recommended drug comprises at least a corresponding one selected from the group consisting of a prohibition reminder, a caution reminder and an allergy reminder;
in a case where the target recommended drug comprises a drug combination, determining incompatibility information of various drugs in the drug combination by inquiring the drug knowledge graph, determining whether an incompatibility is existed between the various drug in the drug combination based on the incompatibility information of the various drugs in the drug combination, and providing an incompatibility reminder in response to that an incompatibility is existed between the various drugs in the drug combination, wherein the check report of the target recommended drug further comprises the incompatibility reminder of the drug combination; and
in a case where the target recommended drug comprises a drug combination, traversing drug combined prescriptions appeared in electronic medical record big data, taking various drugs appeared in the drug combined prescriptions as nodes, setting initial edge-weights between various drugs to 0, and increasing, every time a combination of any two drugs appears in the electronic medical record big data, an edge-weight between the any two drugs, so as to form a network with weights; and determining, based on the network with weights, a safety of the drug combination by a graph search algorithm, wherein the check report of the target recommended drug further comprises a safety determination result of the drug combination.

13. The drug recommendation method according to claim 11, further comprising:
providing the check report of the target recommended drug, while providing the target recommended drug.

14. The drug recommendation method according to claim 1, wherein the providing the target recommended drug comprises:
providing a plurality of medication schemes, wherein each of the plurality of medication schemes comprises at least one drug.

15. The drug recommendation method according to claim 1, further comprising:
constructing the drug knowledge graph.

16. The drug recommendation method according to claim 1, further comprising:
updating the drug knowledge graph based on a selection condition of the target recommended drug.

17. A drug recommendation apparatus, comprising:
a patient information interaction module, configured to obtain patient information;
a candidate drug determination module, configured to determine a candidate drug set based on a drug knowledge graph and the patient information;
a candidate drug scoring module, configured to score each drug in the candidate drug set, and to determine a target recommended drug based on a scoring result; and
a user selection module, configured to provide the target recommended drug.

18. A drug recommendation system, comprising a terminal and a drug recommendation apparatus;
wherein the terminal is configured to send request data to the drug recommendation apparatus; and
the drug recommendation apparatus is configured to:
obtain patient information based on the request data;
determine a candidate drug set based on a drug knowledge graph and the patient information;
score each drug in the candidate drug set, and determine a target recommended drug based on a scoring result; and
provide the target recommended drug to the terminal.

19. An electronic device, comprising:
a memory, configured to store computer readable instructions non-transitorily; and
a processor, configured to execute the computer readable instructions,
wherein upon the computer readable instructions being executed by the processor, the drug recommendation method according to claim 1 is executed.

20. A non-transitory storage medium, storing computer readable instructions non-transitorily, wherein upon the computer readable instructions being executed by a computer, the drug recommendation method according to claim 1 is executed.
